SEQUENCE LISTING (3 sequences)

SEQ ID NO: 1 - protein, 8 aa, Artificial
  an artificially synthesized peptide sequence
SIINFEKL

SEQ ID NO: 2 - protein, 18 aa, Artificial
  an artificially synthesized peptide sequence
LEQLESIINFEKLTEWTS

SEQ ID NO: 3 - protein, 5 aa, Artificial
  an artificially synthesized peptide sequence
MAQVQ

(19) United States
(12) Patent Application Publication
(10) Pub. No.: US 2009/0280511 A1
(43) Pub. Date: Nov. 12, 2009

(54) Methods for selecting candidate peptides presented on the surface of cancer cells following their binding to MHC class I molecules

(75) Inventors: Ichiro Yahara, Nagano (JP); Shigeo Koyasu, Tokyo (JP); Jun Imai, Gunma (JP)

Correspondence Address: LAHIVE & COCKFIELD, LLP;FLOOR 30, SUITE 3000, ONE POST OFFICE SQUARE, BOSTON, MA, 02109, US

(73) Assignees: Medical and Biological Laboratories Co., Ltd., Aichi (JP); Keio University, Tokyo (JP)

(21) Appl. No.: 12/313,855

(22) Filed: Nov. 25, 2008

(30) Foreign Application Priority Data
May 12, 2008 (JP) 2008-125248

(51) Int. Cl. G01N 33/574; G06G 7/60

(52) U.S. Cl. 435/7.23; 703/11

(57) ABSTRACT

An objective of the present invention is to provide methods for selecting candidate peptides presented on the surface of cancer cells following binding to MHC class I molecules, wherein the peptides are involved in the stimulation of T cell immunity (cellular immunity). The present inventors discovered a new principle of MHC I-mediated antigen presentation in which a T cell antigen epitope is more efficiently presented on MHC I molecules when it is positioned at the N-terminal side than when positioned at the C-terminal side.

### FIELD OF THE INVENTION

The present invention relates to methods for selecting candidate peptides involved in the induction of T cell immunity (cellular immunity), which are presented on the surface of cancer cells following their binding to MHC class I molecules.

### BACKGROUND OF THE INVENTION

The living body maintains homeostasis by constantly eliminating non-self invaders and abnormal cells generated within. Cancer cells are also one form of “non-self” generated in the living body. Cellular immunity, which directly targets and destroys cells that have become non-self, plays a major role in its elimination. At this time, cellular immunity distinguishes self/non-self cells by using MHC (major histocompatibility complex) I complexes as markers. In all nucleated cells, MHC I (hereinafter may be referred to as “MHC class I”) binds with peptides that are generated by proteolysis of endogenous proteins in the ubiquitin-proteasome system, and presents them on the cell surface (direct presentation (DP)), providing markers for self and non-self. When peptides on MHC I are derived from self proteins, an immune response is not induced. On the other hand, when peptides derived from proteins that are not present in normal cells are presented due to virus infection or canceration, such non-self protein-expressing cells will be identified as non-self and eliminated. However, there are only a small number of naïve T cells expressing T cell receptors (TCRs) that recognize complexes between MHC I and particular non-self peptides, and they do not exhibit cytotoxicity by themselves. To activate naïve T cells into cytotoxic T lymphocytes (CTLs) (or cytotoxic T cells) and proliferate them to a number sufficient for eliminating nonself cells, both the peptide/MHC I complex, and costimulatory factors expressed only by antigen-presenting cells are essential. Among antigen-presenting cells, dendritic cells (DCs) have a strong ability to present antigens to T cells and activate them, and play an essential role in exerting cellular immunity.

This strong ability of DCs to present antigens and activate cellular immunity has been applied to DC-based cancer immunotherapy (DC therapy), which is drawing much attention as a “science-based therapy”. In DC therapy, DCs are made to present tumor-specific T cell epitopes on MHC I and activate CTLs that recognize them, whereby cancer cells are eliminated. Specifically, DC therapy is a multi-step method comprising the following steps:
1. A tumor-specific antigen is identified in a patient.
2. A T cell epitope is predicted based on the amino acid sequence of the antigen protein and patient's MHC I haplotype;
3. The presence of T cells having TCRs (T cell receptors) that specifically recognize the predicted MHC I-T cell epitope complex is confirmed using MHC tetramer pulsed with the peptide. The peptide is chemically synthesized and complexed with the MHC I molecule (biotinylated at its C terminus) that is synthesized in vitro from mRNA prepared from cDNA. The complex is then allowed to bind to streptavidin (SA) (SA forms a complex with four MHC I molecules having biotin at the C termini). This MHC I-peptide complex is labeled with a fluorescent dye. Since this fluorescent-labeled tetramer binds to CD8⁺ cells having a TCR that recognizes the MHC I-peptide complex, the CD8⁺ cell number can be estimated.
4. Finally, patient-derived DCs are pulsed with the T cell epitope peptide and returned to the patient. When the patient-derived DCs (namely, DCs having the same MHC I haplotype) bound to the peptide are returned to the patient, they stimulate CD8⁺ cells that recognize the peptide and allow them to proliferate and differentiate into CTLs. The CTLs attack cancer cells expressing the MHC I-peptide complex in the patient.

In the above steps, precise prediction of the tumor-specific T cell epitope characterizing non-self cells is crucial.

In general, the efficiency of presentation of a T cell epitope on MHC I is influenced by the following three factors.
1. Expression level and stability of a protein comprising a T cell epitope of interest: the T cell epitope of a protein with higher expression level and less stability is presented more efficiently.
2. Ability of a T cell epitope of interest to bind to MHC I: a T cell epitope more strongly binding to MHC I is presented more efficiently.
3. Competition between a T cell epitope of interest and other T cell epitopes for binding to MHC I: a T cell epitope of a protein with no other potent T cell epitopes is presented efficiently.

However, since tumors are originally from self cells, tumor-specific proteins are encoded by self genes and often expressed in normal cells at low levels. When T cell antigen peptides derived from such self proteins bind strongly to MHC class I molecules and are presented on the surface of self cells according to their numbers, they will be harmful to the body. For this reason, CD8⁺ T cells having a TCR that specifically recognizes complexes between MHC I and T cell antigen epitopes that strongly bind to MHC I are mostly eliminated during the thymic education process (negative selection in the thymus), or otherwise become anergic and show no cytotoxicity by themselves. Therefore, they are not applicable to effective immunotherapy. If CD8⁺ T cells are not educated in the thymus or do not become anergic, they will destroy normal self cells. Accordingly, cancer immunotherapy often uses T cell epitope candidates with moderate MHC I-binding activity. However, there are often many candidates with moderate MHC I-binding activity, and their selection is therefore an extremely inefficient process depending on experience and trial-and-error. If the mechanism behind the presentation efficiency of T cell antigen epitopes is revealed in more detail, T cell antigen epitopes may be more efficiently predicted, and more efficient induction of cancer cell-specific cellular immunity may be possible.

MHC I binds to an oligopeptide (T cell antigen peptide) of 8 to 11 amino acids that results from degradation of intracellular proteins by the ubiquitin-proteasome system, and is expressed on the cell surface (see Yewdell, J. W. et al., Nat. Rev. Immunol. 3, 952-961 (2003); Cresswell, P. et al., Immunol. Rev. 207, 145-147 (2005); Groothuis, T. et al., Curr. Top. Microbiol. Immunol. 300, 127-148 (2005); Shastri, N. et al., Immunol. Rev. 207, 31-41 (2005); Trombetta, E. S. et al., Annu. Rev. Immunol. 23, 975-1028 (2005); and Loureiro, J. et al., Adv. Immunol. 92, 225-305 (2006)). The level of T cell antigen peptide presented is proportional to the expression level and degradation rate of intracellular proteins. T cell antigen peptides should have hydrophobic amino acids at particular positions for binding to MHC I. Most proteins possess such MHC I-binding motifs. Furthermore, human MHC I is polymorphic. As of September, 2007, 1,947 types of HLA-A, B, and C molecules are known (http://www.ebi.ac.uk/imgt/hla/intro.html), each of which having a different MHC I-binding motif. Humans can harbor up to six of these 1,947 MHC I genes (a pair each of the HLA-A, B, and C genes derived from the father and mother). Therefore, peptides derived from almost all proteins expressed in cells are thought to be presented on MHC I. Thus, MHC I and T cell antigen peptides serve as self markers for the immune system by presenting on the cell surface the types and amounts of proteins synthesized in cells. The presentation of the peptides derived from endogenous proteins on MHC I is generally called “direct presentation” (see Janeway, C. et al., Immunobiology: The Immune system in Health and Disease 5th edn. Garland Press, New York (2001)). Cancer cells and virus-infected cells express cancer antigens and viral antigens, respectively, and present peptides derived from them on MHC I. Cytotoxic T cells differentiated from naïve CD8⁺ T cells eliminate non-self cells by recognizing MHC I presenting such non-self antigens (see Janeway, C. et al. supra).

As described above, the MHC I-T cell antigen peptide complex is a marker that the immune system uses to identify self and non-self cells. There are comprehensive databases of all peptides presented on the MHC I, such as the following:

Cancer immunity peptide database
(http://www.cancerimmunity.org/peptidedatabase/Tcellepitopes.htm)

Immune epitope database and analysis resource
(http://beta.immuneepitope.org/intermediateQueryStart.do?dispatch=startIntermediateQ uery, etc.)

In addition, it is also possible to predict antigen peptides of a given protein from its primary structure by searching for MHC I-binding motifs. Such databases include SYFPEITHI (http://www.syfpeithi.de/scripts/MHCServer.dll/home.htm, HLA Peptide Binding Predictions; http://www-bimas.cit.nih.gov/molbio/hla_bind/, etc.).

Meanwhile, T cell antigen peptides are products of the protein quality control mechanism in cells. In addition to proteins reaching the end of their life, polypeptides and proteins that have not acquired proper conformation are also degraded by the cellular protein quality control mechanism. It is recently being suggested that a big part of antigen peptides derive from polypeptides called DRiPs (defective ribosomal products) that have failed to acquire the proper conformation due to various reasons (see Reits, E. A. et al., Nature 404, 774-778 (2000); Schubert, U., et al., Nature 404, 770-774 (2000); Khan, S. et al., J. Immunol. 167, 4801-4804 (2001); Yewdell, J. W. et al., J. Cell Sci. 114, 845-851 (2001); Princiotta, M. F. et al., Immunity 18, 343-354 (2003); Voo, K. S. et al., J. Exp. Med. 199, 459-470 (2004); Qian, S. B. et al., J. Biol. Chem. 281, 392-400 (2006); Qian, S. B. et al., J. Immunol. 177, 227-233 (2006); Yewdell, J. W. et al., Trends Immunol. 270, 368-373 (2006); Eisenlohr, L. C. et al, Nat. Rev. Immunol. 7, 403-10 (2007); and Qian, S. B. et al., J. Biol. Chem. 277, 38818-38826 (2002)). Since DRiPs are degraded soon after their synthesis, their T cell antigen peptides can be presented rapidly to the immune system, particularly to cytotoxic T cells. Considering the presence of this cellular protein quality control mechanism, DRiPs are not necessarily translated up to the C terminus, while proteins reaching the end of their life contain all amino acids from the N to C termini. Although full-length proteins contain an equal amount of the N and C terminal peptides, DRiPs contain more N terminal peptides than C terminal peptides. The exact proportion of DRiP-derived peptides in the total T cell antigen peptides is unknown, but has been pointed out to be not negligible. However, this fact has hardly been considered when selecting T cell antigen peptides used for therapy or such.

### SUMMARY OF THE INVENTION

An objective of the present invention is to provide methods for selecting candidate T cell peptides presented on the surface of cancer cells following their binding to MHC class I molecules.

MHC I-cellular antigen peptide complexes provide information on proteins being synthesized in cells at the time, and serve as immunological self markers. Thus, elucidation of the types and levels of T cell antigen peptides that bind to MHC class I molecules and are presented on the surface of cancer cells is an important task in medical care utilizing the immune system. The present inventors conducted dedicated studies to achieve the objective described above. Specifically, the present inventors quantitatively analyzed the correlation between the expression level of antigen proteins and the amount of presented T cell antigen peptides derived from the antigen proteins to verify the presence of the above-described difference of T cell antigen peptides. As a result, the present inventors discovered that T cell antigen epitopes are more efficiently presented on MHC I molecules when the epitopes are located at the N terminal side than when they are located on the C terminal side, which is a novel rule of MHC I-mediated antigen presentation.

In the present invention, the expression of a fluorescent protein fused with an MHC I epitope (pOV8) and the formation of the MHC I-pOV8 complex were simultaneously measured, and the correlation between the expression of the protein and the presentation of the antigen derived from the protein was directly compared. The result showed that antigen presentation efficiency varied depending on the position of the antigen peptide in the protein (FIG. 1). This N-terminal dominance was observed regardless of the type of cells, type of protein carrying the antigen peptide, alteration of the antigen presentation mechanism by IFN-γ, type of antigen peptide, and such (FIGS. 1 and 2). The result obtained by the present inventors strongly suggests the possibility that N-terminal dominance is a general phenomenon seen in the MHC I-mediated antigen presentation process.

The present invention complements the current lack of information on “qualifications of T cell cancer antigens”. Three requirements need to be fulfilled to qualify as a T cell cancer antigen:

(1) the antigen binds (very strongly) to an MHC class I molecule;
(2) the MHC class I molecule-T cell antigen peptide complex can effectively activate naïve CD8-positive T cells; and
(3) cancer cells express the T cell antigen epitope that binds to the MHC class I molecule.

Of these, (2) has been described in WO 2006/025525 and WO 2006/025526. Although a cancer antigen epitope can be selected based on (1) and (2) alone, whether the antigen epitope is presented on the surface of cancer cells was unknown. However, the present invention has enabled selection of candidate peptides presented on the surface of cancer cells.

The present invention relates to methods for selecting candidate T cell antigen peptides presented on the surface of cancer cells following binding to MHC class I molecules. Specifically, the present invention provides:

[1] a method for determining whether a test peptide is a candidate T cell antigen peptide presented on the surface of a cancer cell following binding to an MHC class I molecule, which comprises the steps of:
(a) providing a test T cell antigen peptide that binds to an MHC class I molecule; and
(b) determining the position of amino acid sequence of said T cell antigen peptide provided in step (a) in the amino acid sequence of a protein comprising the peptide;
wherein, when the position of the amino acid sequence determined in step (b) is within 230 amino acids from the N terminus of the amino acid sequence of the protein, the test peptide is determined to be a candidate T cell antigen peptide presented on the surface of a cancer cell following binding to the MHC class I molecule;
[2] a method for selecting a candidate T cell antigen peptide presented on the surface of a cancer cell following binding to an MHC class I molecule, which comprises the steps of:
(c) determining by the method of [1] whether each of several test peptides is a candidate T cell antigen peptide presented on the surface of a cancer cell following binding to an MHC class I molecule; and
(d) selecting a test peptide that has been determined in step (c) to be a candidate T cell antigen peptide to be presented on the surface of a cancer cell through binding to an MHC class I molecule;
[3] the method of [1] or [2], wherein the test peptide is a partial peptide of a cancer-specific protein;
[4] a method for selecting a candidate T cell antigen peptide presented on the surface of a cancer cell following binding to an MHC class I molecule, which comprises the steps of:
(e) performing an algorithm for predicting MHC class I molecule-binding peptides on the amino acid sequence of a desired protein to identify the amino acid sequence of a peptide that is predicted to bind to an MHC class I molecule;
(f) determining the position of the amino acid sequence identified in step (e) in the amino acid sequence of the protein; and
(g) selecting a peptide comprising the amino acid sequence whose position determined in step (f) is within 230 amino acids from the N terminus of the amino acid sequence of the protein;
[5] a method for identifying a candidate T cell antigen peptide presented on the surface of a cancer cell following binding to an MHC class I molecule, which comprises the step of performing an algorithm for predicting MHC class I molecule-binding peptides on an amino acid sequence positioned within 230 amino acids from the N terminus of a desired protein to identify the amino acid sequence of a peptide that is predicted to bind to an MHC class I molecule, wherein a peptide comprising the amino acid sequence identified in said step is identified as a candidate T cell antigen peptide presented on the surface of a cancer cell following binding to an MHC class I molecule;
[6] the method of [4] or [5], wherein the desired protein is a cancer-specific protein;
[7] a method for identifying a candidate T cell antigen peptide that can be used as a cancer vaccine, which comprises the steps of:
(h) identifying a cancer-specific protein;
(i) identifying the amino acid sequence of a peptide that is predicted to be presented on the surface of a cancer cell through binding to an MHC class I molecule of the same haplotype as that of a patient subjected to cancer treatment or prevention, from the amino acid sequence of the protein identified in step (h);
(j) contacting a peptide comprising the amino acid sequence identified in step (i) with an MHC class I molecule of the same haplotype as that of said subject to form a complex;
(k) contacting the complex formed in step (j) with a CD8-positive T cell isolated from said patient; and
(l) detecting the binding of the complex formed in step (j) to the CD8-positive T cell isolated from the patient;
wherein, when the binding is detected in step (l), a peptide comprising the amino acid sequence identified in step (i) is identified as a candidate T cell antigen peptide that can be used as a cancer vaccine;
[8] a method for identifying a candidate T cell antigen peptide that can be used as a cancer vaccine, which comprises the steps of:
(m) identifying a cancer-specific protein;
(n) performing an algorithm for predicting MHC class I molecule-binding peptides on an amino acid sequence positioned within 230 amino acids from N terminus of the protein identified in step (m) to identify the amino acid sequence of a peptide that is predicted to bind to an MHC class I molecule of the same haplotype as that of a patient subjected to cancer treatment or prevention;
(o) contacting a peptide comprising the amino acid sequence identified in step (n) with an MHC class I molecule of the same haplotype as that of said patient to form a complex;
(p) contacting the complex formed in step (o) with a CD8-positive T cell isolated from the patient; and
(q) measuring the binding of the complex formed in step (o) to the CD8-positive T cell isolated from the patient;
wherein, when the binding is detected in step (q), a peptide comprising the amino acid sequence identified in step (n) is identified as a candidate T cell antigen peptide that can be used as a cancer vaccine; and
[9] a method for identifying a candidate T cell antigen peptide that can be used as a cancer vaccine, which comprises the steps of:
(r) identifying a cancer-specific protein;
(s) performing an algorithm for predicting MHC class I molecule-binding peptides on the amino acid sequence of the protein identified in step (r) to identify the amino acid sequence of a peptide that is predicted to bind to an MHC class I molecule of the same haplotype as that of a patient subjected to cancer treatment or prevention;
(t) determining the position of the amino acid sequence identified in step (s) in the amino acid sequence of the protein identified in step (r);
(u) selecting an amino acid sequence whose position determined in step (t) is within 230 amino acids from the N terminus of the amino acid sequence of the protein identified in step (r), as the amino acid sequence of a peptide to be presented on the surface of a cancer cell through binding to an MHC class I molecule of the same haplotype as that of the patient;
(v) contacting a peptide comprising the amino acid sequence selected in step (u) with an MHC class I molecule of the same haplotype as that of the patient to form a complex;
(w) contacting the complex formed in step (v) with a CD8-positive T cell isolated from the patient; and
(x) detecting the binding of the complex formed in step (v) to the CD8-positive T cell isolated from the patient;
wherein, when the binding is detected in step (x), a peptide comprising the amino acid sequence selected in step (u) is identified as a candidate T cell antigen peptide that can be used as a cancer vaccine.

### BRIEF DESCRIPTION OF THE DRAWINGS

FIG. 1a depicts a set of graphs showing the fluorescence intensity of YFP and the expression level of Kb/pOV8 measured with the 25D1.16 antibody in DC2.4 cells, EL4 cells and MC57G cells in which YFP, pOV8-YFP and YFP-pOV8 were transiently expressed. The YFP fluorescence intensity and the Kb/pOV8 expression level in an equal number of pOV8-YFP-expressing cells and YFP-pOV8-expressing cells were plotted on the horizontal and vertical axes, respectively. The upper row shows DC2.4 cells, the middle row shows EL4 cells and the lower row shows MC57G cells. The first, second, and third columns from the left show YFP alone, pOV8-YFP, and YFP-pOV8, respectively. The rightmost column shows a superimposition of the red-colored plot in the second column from the left and the green-colored plot in the third column. The red-colored sections and green-colored sections are shown by arrows.

FIG. 1b depicts a set of graphs showing the fluorescence intensity of YFP and the expression level of Kb/pOV8 measured with the 25D1.16 antibody in DC2.4 cells, EL4 cells and MC57G cells in which OVA, OVA-YFP and YFP-OVA were transiently expressed. The YFP fluorescence intensity and Kb/pOV8 expression in the equal number of OVA-YFP-expressing cells and YFP-OVA-expressing cells were plotted on the horizontal and vertical axes, respectively. The upper row shows DC2.4 cells, the middle row shows EL4 cells and the lower row shows MC57G cells. The first, second, and third columns from the left show OVA alone, OVA-YFP, and YFP-OVA, respectively. The rightmost column shows a superimposition of the red-colored plot in the second column from the left and the green-colored plot in the third column. The red-colored sections and green-colored sections are shown by arrows.

FIG. 1c depicts a set of graphs showing the fluorescence intensity of Azamigreen and the expression of Kb/pOV8 measured with the 25D1.16 antibody in DC2.4 cells, EL4 cells and MC57G cells in which Azamigreen, pOV8-Azamigreen(AG) and Azamigreen-pOV8 were transiently expressed. The Azamigreen fluorescence intensity and Kb/pOV8 expression level in an equal number of AG-pOV8-expressing cells and pOV8-AG-expressing cells were plotted on the horizontal and vertical axes, respectively. The upper row shows DC2.4 cells, the middle row shows EL4 cells and the lower row shows MC57G cells. The first, second, and third columns from the left show Azamigreen alone, pOV8-Azamigreen, and Azamigreen-pOV8, respectively. The rightmost column shows a superimposition of the red-colored plot in the second column from the left and the green-colored plot in the third column. The red-colored sections and green-colored sections are shown by arrows.

FIG. 1d is a set of graphs showing the fluorescence intensity of YFP and the expression level of Kb/pOV8 measured with the 25D1.16 antibody in DC2.4 cells, EL4 cells and MC57G cells in which YFP-GST, pOV8-YFP-GST and YFP-GST-pOV8 were transiently expressed. The YFP fluorescence intensity and Kb/pOV8 expression level in an equal number of pOV8-YFP-GST-expressing cells and YFP-GST-pOV8-expressing cells were plotted on the horizontal and vertical axes, respectively. The upper row shows DC2.4 cells, the middle row shows EL4 cells and the lower row shows MC57G cells. The first, second, and third columns from the left show YFP-GST alone, pOV8-YFP-GST, and YFP-GST-pOV8, respectively. The rightmost column shows a superimposition of the red-colored plot in the second column from the left and the green-colored plot in the third column. The red-colored sections and green-colored sections are shown by arrows.

FIG. 1e depicts a set of graphs showing analysis of H-2Kb/pOV8 expression using the 25D1.16 antibody, in DC2.4 cells expressing YFP, pOV8-YFP and YFP-pOV8 with or without treatment of IFNγ. The upper row shows the result without IFNγ treatment (IFNγ−) and the middle row shows the result with IFNγ treatment (IFNγ+). The lower row shows a superimposition of the green plot of IFNγ− and the red plot of IFNγ+. The first, second, and third columns from the left show YFP alone, pOV8-YFP, and YFP-pOV8, respectively. The rightmost column shows a superimposition of the red-colored plot in the second column from the left and the green-colored plot in the third column. The red-colored sections and green-colored sections are shown by arrows.

FIG. 2a depicts the results of flow cytometry analysis showing the competition for binding to H-2Kb between pOV8 and GST48 peptide, GST111 peptide or VSV peptide. The amount of H2-Kb/pOV8 on the cell surface was measured using the 25D1.16 antibody (the horizontal axis). The upper row, middle row, and lower row show results of using GST48, GST111, and VSV as a competitive peptide, respectively. The leftmost column shows the result of externally adding pOV8 alone to DC2.4 cells (note: the peptide was directly added to the cells from the outside, not expressed within the cells by protein synthesis), the second column from the left shows GST48, GST111 or VSV in a ratio with pOV8 of 1:9, the third column from the left shows a ratio of 5:5, the fourth column from the left shows a ratio of 9:1, and the rightmost column shows addition of only GST48, GST111 and VSV.

FIG. 2b is a graph showing the level of inhibition of binding of pOV8 to H-2Kb caused by GST48, GST111 or VSV peptide, which was quantified based on the results of FIG. 2a.

FIG. 2c is a set of graphs showing the YFP fluorescence intensity and Kb/pOV8 expression level measured with the 25D1.16 antibody, in DC2.4 cells, EL4 cells and MC57G cells in which pOV8-YFP-GST, pOV8-GST-YFP and YFP-GST-pOV8, or GST-YFP-pOV8 was expressed. The red-colored sections and green-colored sections are shown by arrows.

FIG. 3a shows the process of the time-course experiment on pOV8 epitope expression. DC2.4 cells were transfected with pOV8-YFP and, after 12 hours, washed with acid. Cells which were not washed in acid were sampled as a control. After the acid wash, the cells were sampled at each point of time.

FIG. 3b shows the expression of H-2Kb/pOV8, measured with the 25D1.16 antibody, in DC2.4 cells transfected with pOV8-YFP prepared and sampled in FIG. 3a. The upper row shows the H-2Kb/pOV8 expression. The middle row is a superimposition of the H-2Kb/pOV8 expression increasing over time (the results of the upper row) and the plot for time 0 (green). The lower row is a superimposition of the results of the upper row and the control (green). The leftmost column shows the control cells which were not treated with acid. The second, third, fourth, fifth, and sixth columns from the left and rightmost column show 0, 1, 2, 4, 6, and 12 hours after the acid treatment, respectively. The red-colored sections and green-colored sections are shown by arrows.

FIG. 3c shows the results of the same experiment as FIG. 3b performed using DC2.4 cells transfected with YFP-pOV8. The upper row shows H-2Kb/pOV8 expression. The middle row is a superimposition of the H-2Kb/pOV8 expression increasing over time and the plot for time 0 (green). The lower row is a superimposition of the H-2Kb/pOV8 expression increasing over time and the control (no acid treatment) (green). The leftmost column shows the control. The second, third, fourth, fifth, and sixth columns from the left and the rightmost column show 0, 1, 2, 4, 6, and 12 hours after the acid treatment, respectively. The red-colored sections and green-colored sections are shown by arrows.

FIG. 3d is a graph showing quantification of H-2Kb/pOV8 expression recovery. The expression levels are shown as relative values to the level of H-2Kb/pOV8 in DC2.4 cells expressing pOV8-YFP, which is taken as 100%. The black square represents pOV8-YFP and the white circle represents YFP-pOV8.

FIG. 4a shows the effects of various inhibitors on H-2Kb/pOV8 expression. The DC2.4 cells were washed with acid 12 hours after transfection as in FIG. 3. The inhibitors were added at 0 hours (time 0). After 6 hours, the cells were recovered and examined.

FIG. 4b shows the expression level of H-2Kb/pOV8 in DC2.4 cells measured using 25D1.16 antibody, after the cells were transfected with pOV8-YFP or YFP-pOV8, washed with acid, and cultured with the inhibitors for 6 hours. The upper row shows cells expressing pOV8-YFP, the middle row shows cells expressing YFP-pOV8, and the lower row is a superimposition of the data from the upper and middle rows.

The leftmost column shows cells before the acid wash. The second column from the left shows cells immediately after the acid wash (A.W.). The third column from the left shows cells incubated for 6 hours after addition of 1 μM of MG132. The fourth column from the left shows addition of 0.2 μM of lactacystin, and the rightmost column shows addition of 100 μM of chloroquine. The red-colored sections and green-colored sections are shown by arrows.

FIG. 4c shows the results of adding inhibitors different to those used in FIG. 4b. The upper row shows cells expressing pOV8-YFP, the middle row shows cells expressing YFP-pOV8, and the lower row is a superimposition of the data from the upper and middle rows. The first, second, third, and fourth columns from the left and the rightmost column show the results of adding 10 μg/ml of a-amanitin, 1 μg/ml of anisomycin, 1 μg/ml of emetine, 15 mM of canavanine, and 15 mM of azetidine, respectively. The red-colored sections and green-colored sections are shown by arrows.

FIG. 5 depicts a set of graphs and a photograph showing H2-Kb/pOV8 presentation when YFP, pOV8-YFP and YFP-pOV8 were introduced directly from the outside into the cytosol of DC2.4 cells. The red-colored sections, green-colored sections, and yellow-colored sections are shown by arrows.

FIG. 6 is a schematic diagram showing the origin of pOV8.

FIG. 7a is a photograph showing the expression level of pOV8-YFP and YFP-pOV8 in DC2.4 cells quantified by Western blotting of YFP. Lanes 1, 2, 3, and 4 denote pEF-1a, pEF-1a-MAQVQ-YFP, pEF-1a-MAQVQ-pOV8-YFP, and pEF-1a-MAQVQ-YFP-pOV8, respectively. The amino acid sequence of MAQVQ is shown in SEQ ID NO: 3.

FIG. 7b depicts a graph and a photograph showing the expression level of YFP, pOV8-YFP, and YFP-pOV8 in DC2.4 cells to which cycloheximide was added, quantified by Western blotting. In the graph, the black square represents YFP, the black triangle represents pOV8-YFP and the black circle represents YFP-pOV8.

FIG. 8 depicts graphs showing the affinity of epitopes for MHC class I molecules. The vertical axis on the left graph shows the position of epitope, which is expressed as a relative value where the N-terminus and C-terminus are taken as 0% and 100%, respectively. The vertical axis on the right graph shows the number of amino acids between the epitope and the N-terminus.

FIG. 9 depicts graphs in which the number of amino acids between the epitope and the N-terminus of antigen protein and the affinity for MHC class I molecules are plotted on the vertical and horizontal axes, respectively, for the four types of T cell antigen epitopes (Mutations, Shared Tumor-specific Antigens, Differentiation Antigens and Over-expressed Antigens).

FIG. 10 is a set of graphs showing the affinity of viral antigens for MHC class I molecules. The vertical axis on the left graph shows the position of epitope expressed as a relative value where the N-terminus and C-terminus are taken as 0% and 100%, respectively. The vertical axis on the right graph shows the number of amino acids between the N-terminus and the epitope.

### DETAILED DESCRIPTION OF THE INVENTION

The present invention relates to methods for assessing whether test peptides are candidate T cell antigen peptides presented on the surface of cancer cells following binding to MHC class I molecules.

Specifically, the present invention provides methods for determining whether test peptides are candidate T cell antigen peptides presented on the surface of cancer cells following binding to MHC class I molecules, which comprise the steps of:

(a) providing a test T cell antigen peptide that binds to an MHC class I molecule; and
(b) determining the position of the amino acid sequence of the T cell antigen peptide provided in step (a) in the amino acid sequence of a protein comprising the peptide.

In the methods of the present invention, “presented on the surface of cancer cells” is synonymous with “expressed on the surface of cancer cells” and “displayed on the surface of cancer cells”.

In the methods of the present invention, first, peptides that bind to MHC class I molecules are provided as test peptides.

The “test peptides” used in the present invention are molecules of linear amino acid chains where neighboring amino acid resides are linked via peptide bonds between a-amino groups and carboxyl groups. The peptides may be in an uncharged form or a salt form.

Furthermore, the test peptides may contain hydrophobic amino acids (MHC class I molecule-binding motif) for binding to an MHC class I molecule. The MHC class I molecule-binding motif is restricted only by the haplotype of the MHC class I molecule. The binding motif varies depending on the haplotype. Furthermore, the test peptides used in the present invention may include any type and size of peptides, as long as they can be generated as a result of cleavage and degradation by endopeptidase in the endoplasmic reticulum and the ubiquitin-proteasome system. In most cases, the length of such candidate T cell antigen peptides is 8 to 11 amino acids. Herein, a peptide which is generated as a result of cleavage and degradation by endopeptidase in the endoplasmic reticulum and the ubiquitin-proteasome system and which binds to an MHC class I molecule is referred to as a “T cell antigen peptide”.

In general, molecules composed of a polypeptide chain with a molecular weight of 5,000 or more and those with a molecular weight of less than 5,000 are often distinguished by naming them as “proteins” and “peptides”, respectively (Kagaku Daijiten (Comprehensive Dictionary of Chemistry) Tokyo Kagaku Dozin Co.). Herein, however, such molecules are included in the test peptide of the present invention regardless of the molecular weight, as long as they can be generated as a result of cleavage and degradation by endopeptidase in the endoplasmic reticulum and the ubiquitin-proteasome system. Thus, the test peptide of the present invention also includes so-called “oligopeptides” and “polypeptides”.

Furthermore, the test peptide is not particularly limited, but includes, for example, peptides derived from proteins that are recognized by CTLs. CTLs recognize the MHC class I molecule-T cell antigen peptide complex. The MHC class I molecule is a membrane protein, and therefore the complex is also a membrane protein. The test peptide of the present invention is not limited to peptides of membrane proteins, and includes, for example, fragments of surface proteins on cancer cells, cytosolic proteins, secretory proteins, and mitochondrial proteins. In another embodiment, the peptide includes peptides derived from simple proteins and complex proteins such as nuclear proteins, lipoproteins, glycoproteins, chromogenic proteins, and metalloproteins.

Furthermore, the test peptides of the present invention may be polypeptides called DRiPs, which have failed to acquire the proper tertiary structures due to various reasons. The test peptides of the present invention are preferably T cell cancer antigen peptides.

Herein, peptide portions that bind to an MHC class I molecule to form a complex, and are then presented on the cell surface and recognized by T cells (their TCRs) such as CTLs are referred to as “T cell antigen epitopes” (or, “T cell epitopes”, “T cell antigen peptides” or “T cell peptides” in some cases). In all nucleated cells including cancer cells, a peptide bound to an MHC class I molecule is generally presented on the cell surface regardless of the degree of its affinity for the MHC class I molecule. This degree of this affinity is reflected by the amount of T cell antigen peptide bound to the MHC class I molecule. Meanwhile, the T cell repertoire in an individual undergoes various selections during T cell differentiation. Thus, T cells having TCRs that recognize the peptide that has been presented on the cell surface may not always exist. Therefore, there may be instances where the T cell antigen peptide-MHC class I molecule complex presented on the cell surface is not recognized as an antigen by T cells.

The “candidate T cell antigen peptide presented on the surface of cancer cells” of the present invention is not necessarily the same as a candidate peptide presented on the surface of antigen-presenting cells (for example, dendritic cells). This is because the T cell antigen peptides on the surface of cancer cells are displayed by direct presentation, while the T cell antigen peptides on the surface of antigen-presenting cells are displayed by cross-presentation. For successful CTL therapy or dendritic cell therapy, the T cell antigen peptide needs to be presented on the surface of cancer cells by direct presentation and also presented on antigen-presenting cells by cross-presentation. In other words, T cell antigen peptides that are efficiently expressed (presented) on the cell membrane by the two independent T cell antigen-processing mechanisms, i.e. direct presentation and cross-presentation, are the most suitable candidates in cancer therapy.

The test peptides of the present invention are more preferably partial peptides of cancer-specific proteins. “Cancer-specific proteins” refer to proteins that are expressed only in cancer cells, or proteins whose expression levels are increased in cancer cells as compared to normal cells. The above-described cancer-specific proteins may also include partial peptides thereof.

Herein, “T cell antigen peptides that bind to MHC class I molecules” means T cell antigen peptides that are known to bind to MHC class I molecules, or T cell antigen peptides that have been identified as candidate peptides that bind to MHC class I molecules by using an algorithm for predicting MHC class I molecule-binding peptides.

Such “T cell antigen peptides that are known to bind to MHC class I molecules” can be obtained, for example, by using comprehensive databases that cover all peptides presented on MHC I molecules. Such databases include, for example, Cancer Immunity Peptide Database (http://www.cancerimmunity.org/peptidedatabase/Tcellepitopes.htm) and Immune epitope database and analysis resource (http://beta.immuneepitope.org/intermediateQueryStart.do?dispatch=startIntermediateQuery).

The “algorithm for predicting MHC class I molecule-binding peptides” refers to an algorithm for predicting whether, or how strongly, test peptides bind to the MHC class I. The algorithm of the present invention also includes computer software using the algorithm. Such software includes, for example, SYFPEITHI (http://www.syfpeithi.de/scripts/MHCServer.dll/home.htm), HLA Peptide Binding Predictions (http://www-bimas.cit.nih.gov/molbio/hla_bind), and Bioinformatics & Molecular Analysis Section (BIMAS, http://bimas.dcrt.nih.gov).

In the present method, the next step is to determine where the amino acid sequence of the test peptide predicted to bind to an MHC class I molecule as described above is situated in the amino acid sequence of a protein containing the test peptide.

The peptide position can be determined by methods known to those skilled in the art, for example, by comparing the amino acid sequence of the provided test peptide with the amino acid sequence of the protein containing the peptide predicted to bind to an MHC class I molecule.

The next step of the present method is to determine whether the amino acid sequence examined above is located within the 230 amino acids from the N terminus of the amino acid sequence of the protein. When the test peptide is located within the 230 amino acids, it is determined to be a candidate T cell antigen peptide presented on the surface of cancer cells following binding to the MHC class I molecule.

In the present invention, it is preferred in principle that the amino acid sequence of the peptide is located proximal to the N terminus in the amino acid sequence of the protein containing the peptide. The reason is that, for example, some DRiPs are generated as a result of termination of protein translation at a position more proximal to the N terminus than the 230th amino acid from the N terminus. However, the requirements of the present invention are met if the amino acid sequence of the peptide is located within 230 amino acids from the N terminus. For example, the peptide may be located on the N or C terminus as long as it is located within 230 amino acids from the N terminus, and such peptides may be equally regarded as T cell cancer antigen peptides.

The present inventors discovered that peptides bound to MHC class I molecules were mostly derived from incomplete, freshly-synthesized proteins (called DRiPs), which were generated as a result of premature termination of translation. Thus, the value “230 amino acids” was determined based on the theory that, considering that the average amino acid length of all proteins is 460 amino acids (EMBLE), a peptide located within 230 amino acids from the N terminus may be relatively more often cleaved as compared to a peptide located within the remaining C terminal portion after the 230th amino acid. In fact, most ordinary cancer antigens are located within 230 amino acids from the N terminus.

Meanwhile, whether the length of a protein having a test peptide is 230 amino acids or more or less does not affect the rule that a peptide more proximal to the N terminus binds more preferentially to MHC class I and is presented on the cell membrane.

Herein, the “N terminus of the amino acid sequence of a protein” can be defined as the first amino acid residue at the N terminus of the protein generated by translation. The protein may also comprise the signal sequence of a secretory or membrane protein. In general, the “N terminus of the amino acid sequence of a protein” is often methionine corresponding to the translation initiation codon, but is not limited thereto.

In general, T cell antigen peptides are cleaved at the C terminus of a particular hydrophobic residue by proteasomes, and trimmed at the N terminus by aminopeptidases or such to become a suitable length for binding to MHC class I molecules. Therefore, it is difficult to determine the N terminal amino acid residues of the peptides.

Furthermore, the present invention also provides methods for selecting candidate T cell antigen peptides to be presented on the surface of cancer cells through binding to MHC class I molecules. Specifically, the present invention provides methods for selecting candidate T cell antigen peptides to be presented on the surface of cancer cells through binding to MHC class I molecules, which comprise the steps of:

(c) determining by the determination method described above whether each of several test peptides is a candidate T cell antigen peptide presented on the surface of cancer cells following binding to an MHC class I molecule; and
(d) selecting a test peptide that has been determined in step (c) to be a candidate T cell antigen peptide presented on the surface of cancer cells following binding to the MHC class I molecule.

In the above selection methods of the present invention, first, the above-described determination method of the present invention is performed to determine whether each of several test peptides is a candidate T cell antigen peptide presented on the surface of cancer cells following binding to MHC class I molecules. The next step is to select a test peptide that has been determined to be a candidate peptide presented on the surface of cancer cells following binding to MHC class I molecules.

The present invention also provides methods for selecting candidate T cell antigen peptides presented on the surface of cancer cells following binding to MHC class I molecules. Specifically, the present invention provides methods for selecting candidate T cell antigen peptides presented on the surface of cancer cells following binding to MHC class I molecules, which comprise the steps of:

(e) performing an algorithm for predicting MHC class I molecule-binding peptides on the amino acid sequence of a desired protein to identify the amino acid sequence of a peptide that is predicted to bind to an MHC class I molecule;
(f) determining the position of the amino acid sequence identified in step (e) in the amino acid sequence of the protein; and
(g) selecting a peptide comprising the amino acid sequence that has been determined in step (f) to be located within 230 amino acids from the N terminus of the amino acid sequence of the protein.

In the above selection methods of the present invention, first, an algorithm for predicting MHC class I molecule-binding peptides is performed on the amino acid sequence of a desired protein to identify the amino acid sequence of a peptide predicted to bind to an MHC class I molecule.

In the selection methods, the next step is to determine the position of the identified amino acid sequence in the amino acid sequence of the protein.

The step after that in the selection methods of the present invention is to select peptides whose amino acid sequences have been identified to be located within 230 amino acids from the N terminus of the amino acid sequence of the protein in the above determination. The peptides selected by this procedure are candidate T cell antigen peptides presented on the surface of cancer cells following binding to MHC class I molecules.

Furthermore, the present invention also provides methods for identifying candidate T cell antigen peptides presented on the surface of cancer cells following binding to MHC class I molecules.

In the above identification methods, first, an algorithm for predicting MHC class I molecule-binding peptides is performed on an amino acid sequence positioned within 230 amino acids from the N terminus of a desired protein to detect the amino acid sequence of a peptide that is predicted to bind to MHC class I molecules. The peptide comprising the detected amino acid sequence is identified as a candidate T cell antigen peptide to be presented on the surface of cancer cells through binding to MHC class I molecules.

The above “desired protein” may be any protein; however, cancer-specific proteins are preferred when used in T cell immunotherapy of cancer.

The present invention not only provides methods for identifying candidate peptides used as vaccines, but also guarantees that cancer cells actually express the T cell antigen epitopes. Even if the identified peptide vaccine induces CTL activation, the therapy is not effective when the T cell antigen epitope recognized by the CTLs is not expressed in cancer cells at a sufficiently high level. The essence of the present invention is to predict T cell antigen epitopes that are expressed at higher levels in cancer cells.

Cancer immunotherapy includes vaccine therapy, which is an active immunotherapy. Vaccine therapy includes, for example, immunotherapy using dendritic cells (DC). The immunotherapy using DC (DC therapy) eliminates cancer cells by allowing DCs to present tumor-specific T cell antigen epitopes on MHC I and activate CTLs that recognize the activated DCs to induce an effective anti-tumor immune response. Specifically, DC therapy is a multi-step method comprising the following steps:

1. A patient's tumor-specific antigen is identified.
2. A T cell antigen epitope is predicted based on the amino acid sequence of the antigen protein and patient's MHC I haplotype;
3. The presence of T cells having TCR that specifically recognizes the predicted MHC I-T cell epitope complex is confirmed by using MHC tetramer pulsed with the peptide.
4. Finally, a patient's DCs pulsed with the T cell epitope peptide are returned to the patient, or dendritic cells allowed to incorporate the antigen protein containing the T cell antigen epitope are returned to the patient.

In the above-described DC therapy, immature dendritic cells are preferably used because such dendritic cells actively incorporate antigens from the outside, and then strongly present the incorporated antigens as they mature. Such dendritic cells can be prepared by inducing them using GM-CSF from bone marrow or from peripheral blood CD34-positive precursor cells or monocytes, by directly separating them from peripheral blood mononuclear cells, or the like.

Since effector cells that are effective on cancer cells are CD8-positive cytotoxic T cells (CTLs), CTL activation is also important in DC therapy.

Another embodiment of cancer immunotherapy includes activated lymphocyte therapy, which is one of the passive immune therapies. Activated lymphocyte therapy includes, for example, cytotoxic T cell (CTL) therapy and tumor tissue-infiltrating lymphocyte (TIL) therapy.

Another embodiment of cancer immunotherapy includes peptide vaccine therapy. This therapeutic method comprises administering peptide pharmaceuticals.

The present invention identifies candidate peptides used as effective vaccines in the immunotherapy described above and also antigen proteins that contain the peptides within 230 amino acids from their N termini. In other words, the present invention identifies candidate T cell antigen peptides that are used in DC therapy, CTL therapy, TIL therapy, and peptide vaccine therapy.

Specifically, the present invention provides methods for identifying candidate T cell antigen peptides that can be used as cancer vaccines, which comprises the steps of:

(h) identifying a cancer-specific protein;
(i) identifying the amino acid sequence of a peptide that is predicted to be presented on the surface of cancer cells following binding to an MHC class I molecule of the same haplotype as that of a subject in the treatment or prevention of the cancer, from the amino acid sequence of the protein identified in step (h);
(j) contacting a peptide comprising the amino acid sequence identified in step (i) with an MHC class I molecule of the same haplotype as that of the subject to form a complex;
(k) contacting the complex formed in step (j) with a CD8-positive T cell isolated from the subject; and
(l) measuring the binding between the complex formed in step (j) and the CD8-positive T cell isolated from the subject;
wherein, when the binding is detected in step (l), a peptide comprising the amino acid sequence identified in step (i) is identified as a candidate T cell antigen peptide that can be used as a cancer vaccine.

In the above identification methods, first, a cancer-specific protein is identified. Cancer-specific proteins can be identified by known methods based on transcriptome or proteome analysis, for example, by methods using DNA microarray analysis, analysis methods using protein chip systems, and two-dimensional gel electrophoresis.

Then, the amino acid sequences of peptides that are predicted to be presented on the surface of cancer cells following binding to MHC class I molecules of the same haplotype as that of a subject in cancer treatment or prevention are detected from the amino acid sequence of the identified protein.

In the next step of the above methods, a peptide comprising the identified amino acid sequence is contacted with the MHC class I molecule of the same haplotype as that of the subject to form a complex. Then, CD8-positive T cells isolated from the subject are contacted with the formed complex, and their binding is measured. The binding between the complex and CD8-positive T cells can be measured by known methods, specifically, by using an MHC-tetramer assay. It is also possible to quantify (count) the number of activated CD8-positive T cells using an ELISPOT assay, measure the degree of direct cytotoxicity using a chromium release assay, assess the degree of activation of CD8-positive cells using intracellular cytokine staining, or such (Current Protocols in Immunology, Edited by: John E. Coligan, Ada M. Kruisbeek, David H. Margulies, Ethan M. Shevach, Warren Strober, 6.19 ELISPOT Assay to Detect Cytokine-Secreting Murine and Human Cells, 6.24 Detection of Intracellular Cytokines by Flow Cytometry, published by John Wiley & Sons, Inc.).

Furthermore, when the binding is detected, a peptide comprising the identified amino acid sequence described above is identified as a candidate T cell antigen peptide that can be used as a cancer vaccine.

Furthermore, the present invention also provides an alternative embodiment of methods for identifying candidate T cell antigen peptides that can be used as vaccines. Specifically, the present invention provides methods for identifying candidate T cell antigen peptides that can be used as vaccines, which comprises the steps of:

(m) identifying a cancer-specific protein;
(n) performing an algorithm for predicting MHC class I molecule-binding peptides on an amino acid sequence positioned within 230 amino acids from the N terminus of the protein identified in step (m) to identify the amino acid sequence of a peptide that is predicted to bind to an MHC class I molecule of the same haplotype as that of a subject in cancer treatment or prevention;
(o) contacting a peptide comprising the amino acid sequence identified in step (n) with an MHC class I molecule of the same haplotype as that of the subject to form a complex;
(p) contacting the complex formed in step (o) with a CD8-positive T cell isolated from the subject; and
(q) measuring the binding between the complex formed in step (o) and the CD8-positive T cell isolated from the subject;
wherein, when the binding is detected in step (q), a peptide comprising the amino acid sequence identified in step (n) is identified as a candidate T cell antigen peptide that can be used as a cancer vaccine.

In the above identification methods, first, a cancer-specific protein is identified. Then, an algorithm for predicting MHC class I molecule-binding peptides is performed on the amino acid sequence positioned within 230 amino acids from the N terminus of the identified protein to detect the amino acid sequence of a peptide predicted to bind to an MHC class I molecule of the same haplotype as that of a subject in cancer treatment or prevention. Then, the MHC class I molecule of the same haplotype as that of the subject is contacted with a peptide comprising the identified amino acid sequence to form a complex. Next, the complex is contacted with CD8-positive cells isolated from the subject. The binding between the complex and CD8-positive T cells isolated from the subject is then measured.

Furthermore, the present invention also provides another alternative embodiment of methods for identifying candidate T cell antigen peptides that can be used as vaccines. Specifically, the present invention provides methods for identifying candidate T cell antigen peptides that can be used as cancer vaccines, which comprises the steps of:

(r) identifying a cancer-specific protein;
(s) performing an algorithm for predicting MHC class I molecule-binding peptides on the amino acid sequence of the protein identified in step (r) to identify the amino acid sequence of a peptide that is predicted to bind to an MHC class I molecule of the same haplotype as that of a subject in cancer treatment or prevention;
(t) determining the position of the amino acid sequence identified in step (s) in the amino acid sequence of the protein identified in step (r);
(u) selecting an amino acid sequence whose position determined in step (t) is located within 230 amino acids from the N terminus of the amino acid sequence of the protein identified in step (r), as the amino acid sequence of a peptide presented on the surface of a cancer cell following binding to an MHC class I molecule of the same haplotype as that of the subject;
(v) contacting a peptide comprising the amino acid sequence selected in step (u) with an MHC class I molecule of the same haplotype as that of the subject to form a complex;
(w) contacting the complex formed in step (v) with a CD8-positive T cell isolated from the subject; and
(x) measuring the binding between the complex formed in step (v) and the CD8-positive T cell isolated from the subject;
wherein, when the binding is detected in step (x), a peptide comprising the amino acid sequence selected in step (u) is identified as a candidate T cell antigen peptide that can be used as a cancer vaccine.

In the above methods, first, a cancer-specific protein is identified. Then, an algorithm for predicting MHC class I molecule-binding peptides is performed on the amino acid sequence of the identified protein to identify the amino acid sequence of a peptide predicted to bind to an MHC class I molecule of the same haplotype as that of a subject in cancer treatment or prevention. Next, the position of the identified amino acid sequence in the amino acid sequence of the identified cancer-specific protein is determined. Then, the amino acid sequence whose position determined is within 230 amino acids from the N terminus of the amino acid sequence of the identified cancer-specific protein as described above is selected as the amino acid sequence of a peptide to be presented on the surface of cancer cells through binding to the MHC class I molecule of the same haplotype as that of the subject. Next, a peptide comprising the amino acid sequence selected as described above is contacted with the MHC class I molecule of the same haplotype as that of the subject to form a complex. The formed complex is then contacted with CD8-positive T cells isolated from the subject. This step is followed by detecting the binding between the formed complex and CD8-positive T cells isolated from the subject. When binding is detected, the peptide presented on the surface of cancer cells following binding to the MHC class I molecule of the same haplotype as that of the subject is identified as a candidate T cell antigen peptide that can be used as a cancer vaccine.

Furthermore, the present invention also provides immunotherapy using the above-described candidate T cell antigen peptides that can be used as vaccines. Specifically, antigen-presenting cells that present candidate peptides obtained by the methods of the present invention can be used as effective vaccines in active immunotherapy. The reason of using the adjective “effective” herein is that, even when CTLs that recognize a desired MHC I-T cell antigen epitope are induced during immunotherapy, target cancer cells often do not express the MHC I-T cell antigen epitope. As a matter of course, immunotherapy fails in such cases. Antigen-presenting cells that present candidate peptides refer to:

1. antigen-presenting cells pulsed with a candidate peptide, which are obtained by incubating antigen-presenting cells with a candidate peptide for 30 minutes to one hour in an appropriate culture medium;
2. candidate-peptide-presenting cells, which are obtained by allowing antigen-presenting cells to incorporate a T cell antigen protein containing the candidate peptide in an appropriate culture medium;
3. antigen-presenting cells allowed to present a candidate peptide by introducing a nucleic acid encoding the candidate peptide into the cells;
4. artificial antigen-presenting constructs having the ability to present antigens; or such.

The term “antigen-presenting cell” refers, for example, to dendritic cells, B cells, macrophages, certain types of T cells, and such, but means cells that express on their surface an MHC class I molecule to which a desired candidate peptide can bind, and have the ability to stimulate CTLs. Artificial antigen-presenting constructs having the ability to present antigens can be prepared, for example, by the following procedure: MHC class I molecule-candidate peptide complexes are immobilized onto a lipid bilayer, plastic or latex beads, or such; and then, co-stimulatory molecules such as CD80, CD83, or CD86, which can stimulate CTLs, or antibodies or the like acting agonistically on CD28, a T-cell ligand to which the co-stimulatory molecules bind, are immobilized (Oelke M, Maus M V, Didiano D, June C H, Mackensen A, Schneck J P., Ex vivo induction and expansion of antigen-specific cytotoxic T cells by HLA-Ig-coated artificial antigen-presenting cells., Nat Med. 2003; 9:619-624; Walter S, Herrgen L, Schoor O, Jung G, Wernet D, Buhring H J, Rammensee H G, Stevanovic S., Cutting edge: predetermined avidity of human CD8 T cells expanded on calibrated MHC/anti-CD28-coated microspheres., J. Immunol. 2003; 171:4974-4978; Oosten L E, Blokland E, van Halteren A G, Curtsinger J, Mescher M F, Falkenburg J H, Mutis T, Goulmy E., Artificial antigen-presenting constructs efficiently stimulate minor histocompatibility antigen-specific cytotoxic T lymphocytes., Blood. 2004; 104:224-226).

Vaccines containing candidate T cell antigen peptides obtained by the methods of the present invention can be prepared by methods known in the art. Such vaccines include, for example, pharmaceuticals such as injection agents and solid agents containing as an active ingredient candidate T cell antigen peptides of the present invention.

Immunotherapy includes passive immunotherapy as well as the active immunotherapy described above. Passive immunotherapy includes, for example, CTL therapy and TIL therapy.

Specifically, CTL therapy using candidate T cell antigen peptides obtained by the methods of the present invention can be performed by administering the candidate peptides to healthy persons or cancer patients and allowing cancer-specific CTLs to grow in their bodies. This therapy may be useful in preventing or treating cancer.

Immunotherapy can be carried out using only one type of candidate T cell antigen peptide, or using two or more types of candidate T cell antigen peptides combined and mixed depending on the purpose of vaccination. Thus, the present invention provides vaccines for treating or preventing cancer, which comprise as active ingredients candidate T cell antigen peptides of the present invention.

Herein, the term “vaccines” can also be expressed as “active immunotherapeutic agents”, “immunotherapeutic agents”, or “cancer therapeutic agents”. Moreover, the “therapeutic agents” can be herein referred to as “pharmaceuticals”, “pharmaceutical compositions”, “therapeutic medicines”, or the like.

Furthermore, the present invention also includes methods for producing cancer-specific CTLs that are used in the CTL therapy described above.

Herein, cancers include pathological conditions of blood and hematopoietic tissues (leukemia and lymphoma) and solid tumors. Solid tumors include carcinomas and sarcomas. Specifically, included are melanoma, breast cancer, and prostate cancer, but are not limited thereto.

The present invention enables selection of T cell antigen peptides in view of actual biological reaction processes, and thereby reduces the number of trial-and-error attempts for selecting MHC I epitopes truly reactive to CTLs. For example, the present inventors consider that this N-terminal dominance is true for at least antigens derived from cancer-specific proteins. The results obtained by the present inventors indicate that the position of candidate T cell antigen epitope in a protein is important in MHC I-mediated antigen presentation.

Although conventional cancer vaccines contain cancer cell-specific T cell antigen peptides, whether they are expressed abundantly on cancer cells was hardly taken into account.

On the other hand, the present invention identifies candidate T cell antigen peptides presented on the surface of cancer cells following binding to MHC class I molecules. Identified candidate T cell antigen peptides can be used to develop more effective cancer vaccines.

All prior art documents cited herein are incorporated herein by reference.

### EXAMPLES

Herein below, the present invention will be specifically described with reference to Examples, but it is not to be construed as being limited thereto.

### Example 1

### 1-1

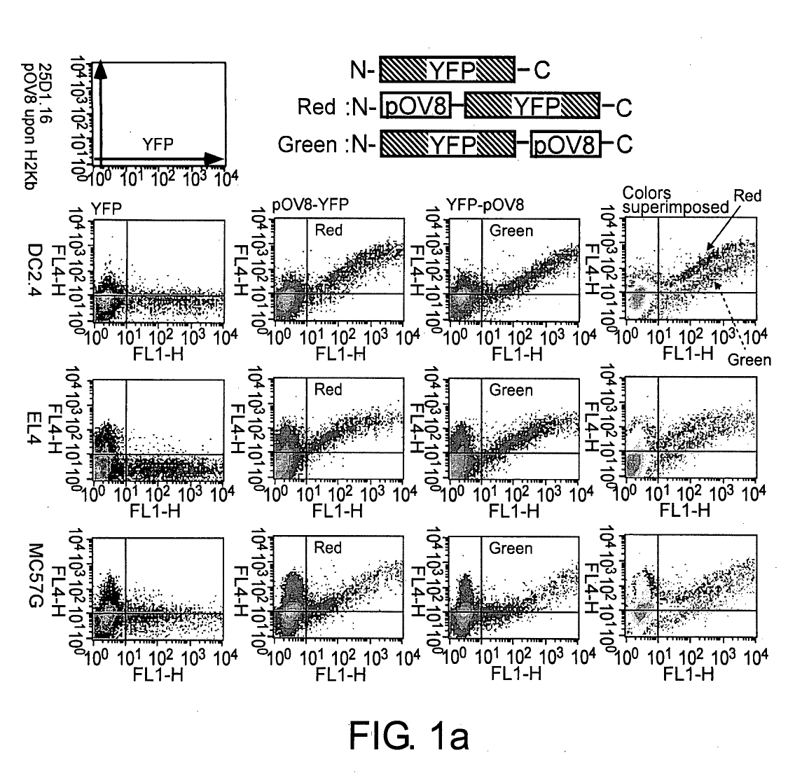

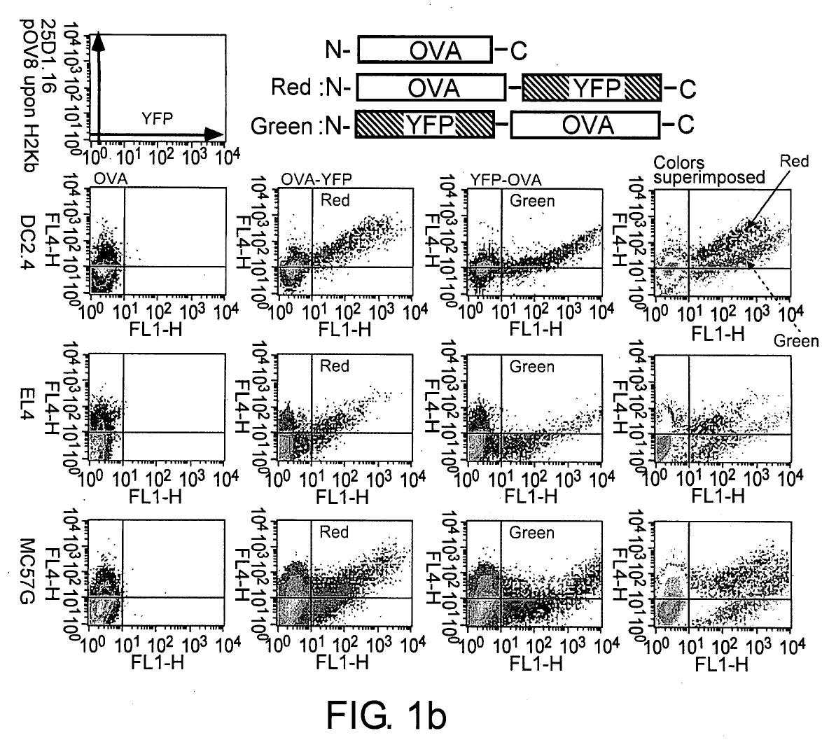

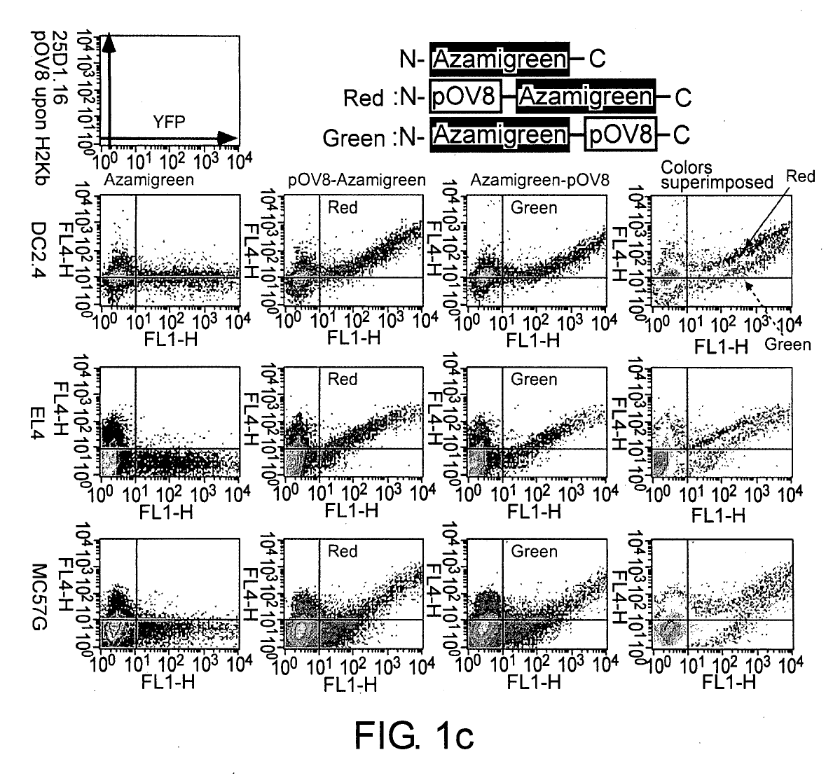

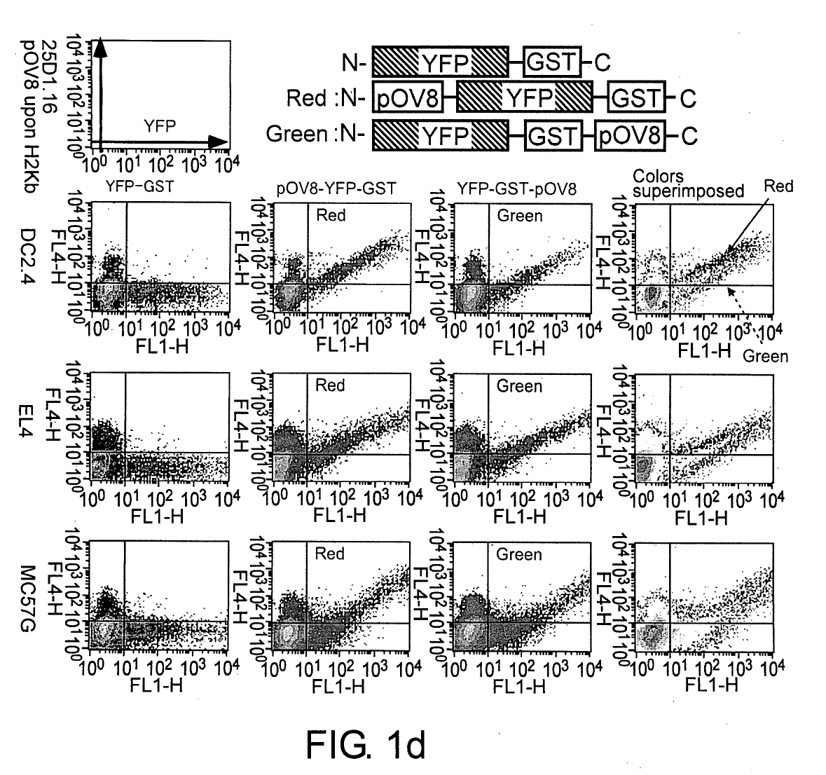

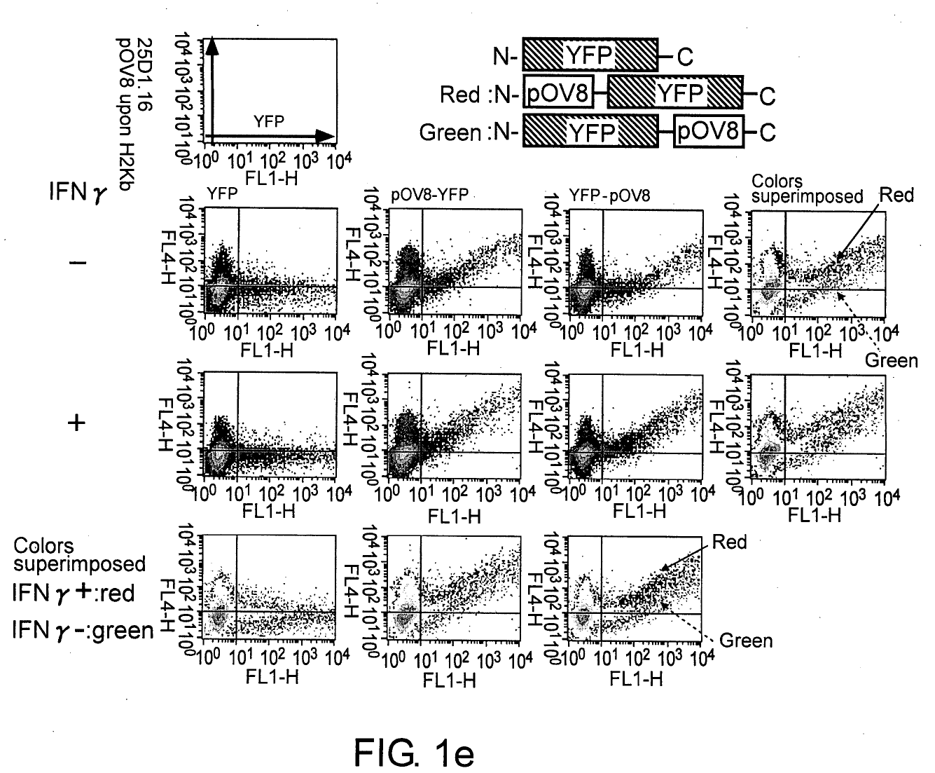

pOV8-YFP and YFP-pOV8 were transiently expressed in DC2.4 cells (FIG. 1a: columns 2 and 3). 25D1.16 antibody was used to measure the YFP fluorescence intensity and the expression level of Kb/pOV8 in the respective cells. The YFP fluorescence intensity and the expression level of Kb/pOV8 in pOV8-YFP-expressing cells and YFP-pOV8-expressing cells, were plotted on the horizontal and vertical axes, respectively.

Expression method: Five flanking amino acids were added to the N-terminus and C-terminus of pOV8 (SIINFEKL/SEQ ID NO: 1) and expressed as a fusion protein with YFP (LEQLESIINFEKLTEWTS-YFP or YFP-LEQLESIINFEKLTEWTS). The amino acid sequence LEQLESIINFEKLTEWTS is shown in SEQ ID NO: 2.

Cells used for expression: The first, second, and third rows of FIG. 1a represent DC2.4 (H-2Kb mouse dendritic cell line), EL4 (H-2Kb mouse leukemia cell line), and MC57G (H-2Kb mouse fibrosarcoma cell line), respectively.

The results are shown in FIG. 1a. The X axis shows the fluorescence intensity of expressed YFP. The Y axis shows the expression level of Kb/pOV8 measured using the 25D1.16 antibody. Individual dots correspond to respective cells.

Each plot showed a roughly straight line sloping upward to the right. In other words, the expression level of the fusion protein measured by YFP fluorescence was directly proportional to the amount of H2-Kb/pOV8 presented, as determined by the 25D1.16 antibody. Superimposition of the two plots (the rightmost column in FIG. 1a) showed that the pOV8-YFP plot was higher than the YFP-pOV8 plot. That is to say, pOV8-YFP caused a higher level of Kb/pOV8 expression than YFP-pOV8 when the same amount of protein was expressed (at the same position on the horizontal axis). Put another way, these results show that the epitope (pOV8) is more easily expressed on the H-2Kb molecule when located at the N-terminus of YFP than when located on the C-terminus of YFP. Hereafter this tendency is referred to as “N-terminal epitope dominance”.

The exact same results were obtained in three types of cell lines (DC2.4, EL4, and MC57G).

### 1-2

A control experiment confirmed the following points:

(1) When only YFP was expressed, the plot only developed in a horizontal direction. (the leftmost column in FIG. 1a).

(2) When only OVA was expressed, the plot only developed in a vertical direction. (the leftmost column in FIG. 1b).

### 1-3

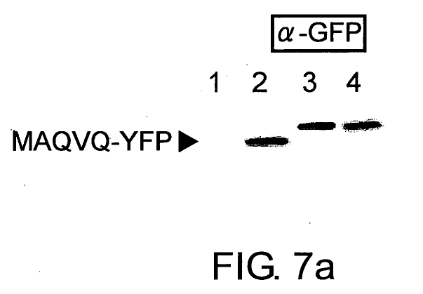

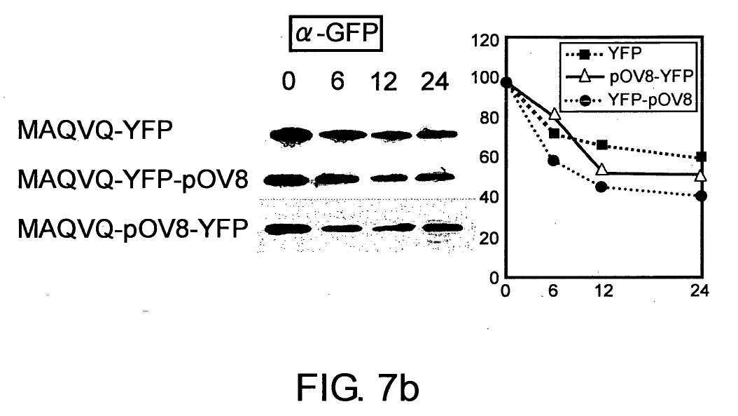

Western blot quantification of YFP showed that the expression levels of pOV8-YFP and YFP-pOV8 in DC2.4 cells were substantially equal on the total protein basis (FIG. 7a).

This result demonstrates that the difference in the expression of pOV8 antigen does not result from a difference in the level of protein expression.

### 1-4

DC2.4 cells were transfected with YFP, pOV8-YFP and YFP-pOV8. Transfection efficiency was confirmed to be the same for each Cycloheximide, a protein synthesis inhibitor, was added to the cells and incubated. At designated time periods, cells were removed to prepare a cell extract. Equal quantities of protein in the cell extracts were separated using SDS-PAGE and analyzed by Western blotting using an anti-YFP antibody.

The results showed that intracellular pOV8-YFP and YFP-pOV8 were degraded at substantially the same rate and, at least, pOV8-YFP was not more rapidly degraded than YFP-pOV8. These results demonstrate that the difference in epitope presentation is not attributed to a difference in the bulk stability of both proteins expressed in the cells (FIG. 7b).

### Example 2

### Generality of N-terminal Epitope Dominance (1)

Experiments were conducted using a YFP protein fused with the entire length of OVA instead of pOV8.

OVA-YFP and YFP-OVA were transiently expressed in DC2.4 cells, and the YFP fluorescence intensity and the expression level of Kb/pOV8 in the respective cells were measured using the 25D1.16 antibody. Superimposition of the two plots showed that the amount of pOV8 presented on H-2Kb was larger when OVA was fused to the N-terminus of YFP than when fused to the C-terminus (the rightmost column of FIG. 1b).

### Example 3

### Generality of N-terminal Epitope Dominance (2)

Experiments were conducted using other proteins fused with pOV8.

[3-1]

pOV8-Azamigreen (AG) and Azamigreen-pOV8 were transiently expressed in DC2.4 cells, and the Azamigreen fluorescence intensity and the expression level of H-2Kb/pOV8 in the respective cells were measured using the 25D1.16 antibody. The Azamigreen fluorescence intensity and the H-2Kb/pOV8 expression in cells expressing AG-pOV8 and the cells expressing pOV8-AG, were plotted on the X axis and Y axis, respectively.

Similar to the results for the YFP fusion protein, the results showed that H-2Kb/pOV8 expression was higher when pOV8 was fused to the N-terminus of the Azamigreen protein than when fused to the C-terminus (FIG. 1c). [3-2]

Further experiments were conducted using YFP-GST instead of YFP, noting the following points:

(1) It has been suggested that GST has epitopes that bind more strongly to H-2Kb than the sequences in pOV8 or YFP (data from databases).
(2) GST is not a fluorescent protein, therefore intracellular expression was monitored using a YFP-GST fusion protein.

The results showed that presentation of pOV8 was stronger when pOV8 was fused to the N-terminus of YFP-GST (pOV8-YFP-GST) than when fused to the C-terminus (YFP-GST-pOV8) (the rightmost column in FIG. 1d). Thus, the N-terminal epitope dominance was established in this case as well. Although pOV8 presentation was weakened due to competition with many epitope sequences in GST that strongly bind to H-2Kb, this did not affect the N-terminal epitope dominance.

[3-3]

Further Experiments were conducted using another fluorescent protein, Kaede, instead of YFP. pOV8-Kaede and Kaede-pOV8 were respectively expressed in DC2.4 cells and the level of H-2Kb/pOV8 presentation was examined. Again, the H-2Kb/pOV8 expression showed a higher level when pOV8 was fused to the N-terminus of Kaede than when fused to the C-terminus (data not shown).

### Example 4

### Generality of N-terminal Epitope Dominance (3)

Experiments were conducted using cells other than DC2.4 cells.

[4-1]

Experiments same as those in [1-1] were repeated after changing the cells used for expression to EL4 cells (T lymphoma cell line). Similar to the results for DC2.4 cells, the pOV8-YFP plot was higher than the YFP-pOV8 plot. Thus, N-terminal epitope dominance was confirmed for EL4 cells (middle row in FIG. 1a) as well.

[4-2]

Next, OVA-YFP and YFP-OVA were respectively expressed in EL4 cells. Similar to the results for DC2.4 cells ([2] above), pOV8 presentation was stronger when OVA-YFP was expressed than when YFP-OVA was expressed. Thus, N-terminal epitope dominance was maintained (middle row in FIG. 1b).

[4-3]

Further, pOV8-AG and AG-pOV8 were compared in EL4 cells (similar to [3-1] above).

As a result, pOV8 presentation showed a higher level when pOV8-AG was expressed than when AG-pOV8 was expressed. Thus, N-terminal epitope dominance was maintained (middle row in FIG. 1c).

[4-4]

pOV8-YFP-GST and YFP-GST-pOV8 were compared in EL4 cells (similar to [3-2] above).

As a result, pOV8 presentation showed a higher level when pOV8-YFP-GST was expressed than when YFP-GST-pOV8 was expressed. Thus, N-terminal epitope dominance was maintained (middle row in FIG. 1d).

[4-5]

Experiments corresponding to [4-1]-[4-4] were conducted using MC57G (fibrosarcoma tumor cell line).

Similar to the above, pOV8 presentation showed a higher level when pOV8 was positioned in the N-terminus than when positioned in the C-terminus. Thus, N-terminal epitope dominance was confirmed for MC57G cells (lower row in FIGS. 1a, 1b, 1c and 1d) as well.

### Example 5

Experiments were conducted to test epitopes that compete with pOV8 for binding to H-2Kb.

The GST protein contains sequences termed GST48 and GST111, which are known to have a high affinity to H-2Kb.

Although there are no antibodies which recognize GST epitope-H-2Kb complexes, the expression level of H-2Kb/GST epitope can be indirectly estimated. When these GST sequences coexist with pOV8, they may compete with each other for binding to H-2Kb. Specifically, when pOV8 and GST are coexpressed in cells, high expression of H-2Kb/GST epitope will reduce the expression of H-2Kb/pOV8, and low expression of H-2Kb/GST epitope will increase the expression of H-2Kb/pOV8. Consequently, it is hypothesized that the amount of H-2Kb/GST epitope presented at the cell surface can be estimated from the corresponding decrease in the amount of coexpressed H-2Kb/pOV8. (Supplementary explanation: Assume that when pOV8/YFP is taken up by DC2.4 cells, 1/10 of total H-2Kb molecules are expressed as H-2Kb/pOV8. Next, assume that pOV8-YFP-GST is taken up, and that GST peptides such as GST48 and GST111 can form complexes with ⅕ of overall H-2Kb molecules in the absence of pOV8 expression. When pOV8 and GST-derived peptides coexist, the ratio of H-2Kb/pOV8 may be simply calculated as 0.1/1.2=0.083. In other words, the ratio of pOV8 expression is reduced from 0.1 to 0.083 by coexisting GST sequences. This reduction ratio increases with the increase of GST-derived antigenic peptide expression). Based on this principle, the level of H-2Kb/pOV8 expression was compared between cells expressing pOV8-YFP-GST and pOV8-GST-YFP.

[5-1]

First, competitive binding by pOV8 and GST48 or GST111 with H-2Kb was directly analyzed. pOV8 and GST48 peptides were mixed at various ratios to a total of 50 μM and added to DC2.4 cells. These peptides bound directly to H-2Kb expressed on the cellular surface (more precisely, they replaced peptides binding with a weak affinity to H-2Kb. The amount of H-2Kb/pOV8 was measured using 25D1.16.

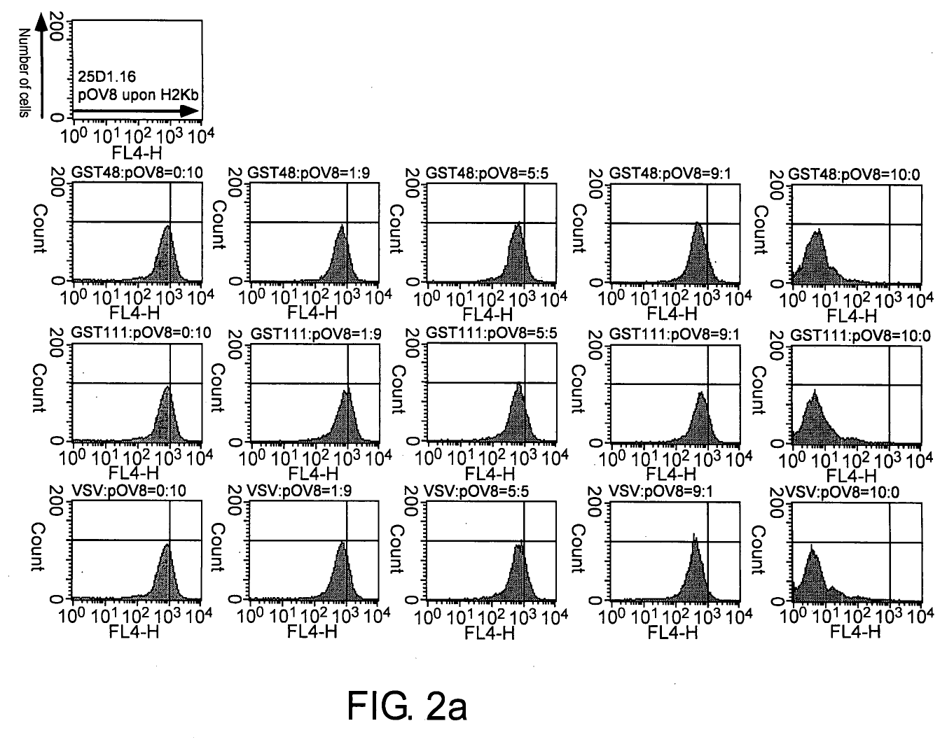

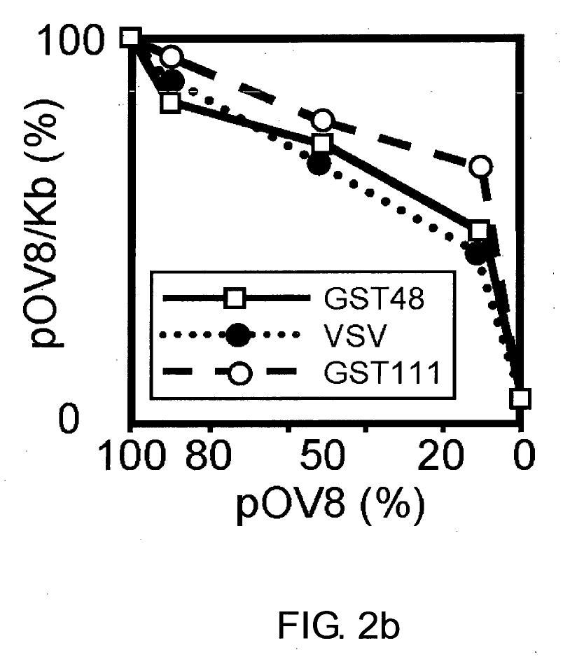

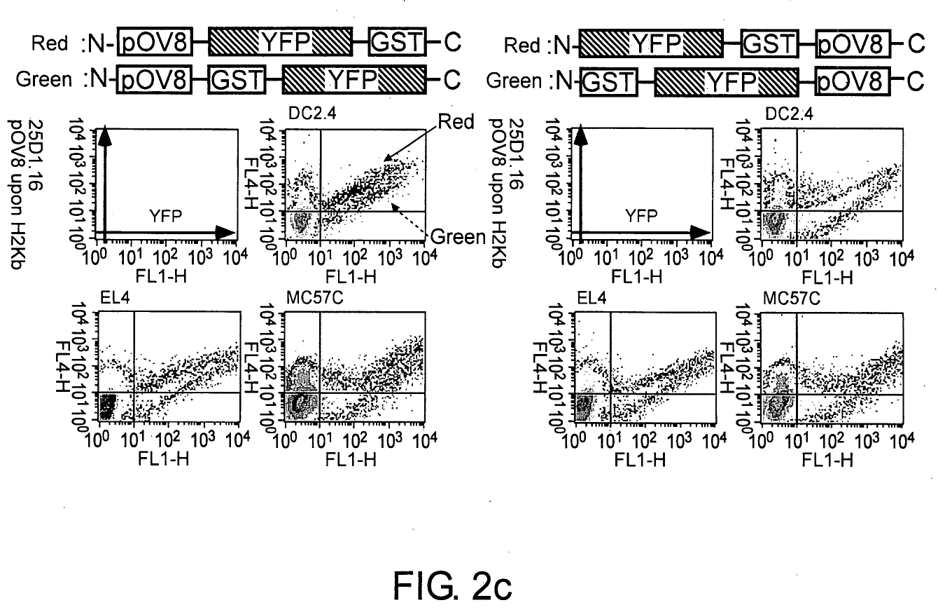

The results indicated that the amount of H-2Kb/pOV8 decreased with the increase of GST48 ratio (FIG. 2a).

H-2Kb/pOV8 levels were measured when GST48 and pOV8 were mixed in ratios of 1:9, 5:5, and 9:1 (across the columns to the right in FIG. 2a) relative to adding only pOV8 (the leftmost column of FIG. 2a). The rightmost column in FIG. 2a shows the addition of GST48 only. It should be noted that both of the above peptide epitopes do not replace all other peptides binding to H-2Kb on the cell membrane (do not replace peptides having a higher affinity).

The results of competitive binding between the GST111 peptide and pOV8 are shown in FIG. 2a.

Similar competitive binding experiments were conducted for VSV peptides, which display a strong affinity for H-2Kb. The results are shown in the lower row of FIG. 2a.

The inhibition of the binding of pOV8 to H-2Kb by GST48, GST111 and VSV peptides were quantified and shown graphically (FIG. 2b).

[5-2]

The competition for binding to H-2Kb between pOV8 and GST was also examined using cells expressing both peptides, instead of directly adding them to the cells.

As shown by the plot in the left of FIG. 2c, the H-2Kb/pOV8 expression was lower when GST was fused with the N-terminus of YFP than when GST was fused with the C-terminus. In other words, GST competed more strongly with pOV8 for binding to H-2Kb when fused with the YFP N-terminus than when fused with the C-terminus.

These results obviously show that more GST peptides were bound to H-2Kb when GST was fused with the N-terminus of YFP than when fused to the C-terminus. Consequently, the N-terminal epitope dominance was demonstrated in this case also. The results were basically the same whether competitive pOV8 was fused at the farthest N-terminus or the farthest C-terminus (the right figure in FIG. 2c).

The use of EL4 cells or MC57G cells basically yielded the same results (data not shown).

### Example 6

MHC class I antigen expression is known to increase when cells are treated with IFNγ. Experiments were conducted to test whether IFNγ treatment altered the effect of pOV8 position in the fusion protein (N-terminal or C-terminal) on the expression of H-2Kb/pOV8.

First, DC2.4 cells expressing pOV8-YFP and YFP-pOV8 were respectively treated with IFNγ and analyzed for H-2Kb/pOV8 expression using the 25D1.16 antibody.

The upper row in FIG. 1e shows the results in the absence of IFNγ treatment and the middle row shows the results with IFNγ treatment. As shown by the rightmost column, independently of IFNγ treatment, H-2Kb/pOV8 expression was higher when pOV8-YFP was expressed than when YFP-pOV8 was expressed, indicating that N-terminal epitope dominance was maintained.

The lower row in FIG. 1e shows comparison of the results with or without IFNγ treatment in cells expressing:
YFP alone (leftmost column),
pOV8-YFP (second column from the left), or
YFP-pOV8 (second column from the right), where the plot of no IFNγ treatment has been colored green and superimposed onto the plot of IFNγ treatment. H-2Kb/pOV8 expression was increased by IFNγ treatment whether pOV8-YFP or YFP-pOV8 was expressed.

Cellular IFNγ treatment causes an increase in class I antigen expression, and the enormous immunological significance of this phenomenon has been elucidated. The principle of N-terminal epitope dominance should play a significant role in direct antigen presentation in the living body, since it brings about an expression difference similar to that which results from the presence or absence of IFNγ treatment. Table 1 (comparison of the effect of N-terminal epitope dominance and the effect of IFNγ treatment on cell surface H-2Kb/pOV8 expression) shows a comparison of the increase ratio in class I molecule-T cell antigenic peptide complexes resulting from IFNγ treatment and the increase ratio in class I molecule-T cell antigenic peptide complexes when the antigenic peptide is located more towards the N-terminus than the C-terminus.

TABLE 1
IFNγ+/IFNγ−

| | Whole | | at 10² fluorescent units | |
|---|---|---|---|---|
| | p0V8-YFP | YFP-p0V8 | p0V8-YFP | YFP-p0V8 |
| IFNγ+/IFNγ− | 4.15 ± 0.21 | 4.06 ± 0.24 | 3.22 ± 0.29 | 3.28 ± 0.33 |

p0V8-YFP/YFP-p0V8

| | Whole | | at 10² fluorescent units | |
|---|---|---|---|---|
| | IFNγ+ | IFNγ− | IFNγ+ | IFNγ− |
| p0V8-YFP/ | 1.68 ± 0.06 | 1.72 ± 0.14 | 2.20 ± 0.12 | 2.24 ± 0.19 |
| YFP-p0V8 | | | | |

Table 1 shows the results of quantifying H-2Kb/pOV8 expression with respect to pOV8-YFP or YFP-pOV8 in the presence or absence of IFNγ treatment. In the table, the term “whole” means the sum of H-2Kb/pOV8 expression for all cells expressing YFP. The term “at 10² fluorescent units” means H-2Kb/pOV8 expression for cells expressing 10² fluorescent units of YFP. For “whole” (for 10² fluorescent units), both pOV8-YFP and YFP-pOV8 showed an increase in H-2Kb/pOV8 expression by approximately 4-fold (approximately 3.2-fold) as a result of IFNγ treatment. The lower table in Table 1 shows a comparison of N-terminal epitope position with C-terminal epitope position. Irrespective of IFNγ treatment, pOV8-YFP for “whole” (10² fluorescent units) showed approximately 1.7-fold (2.2-fold) H-2Kb/pOV8 antigen expression compared to YFP-pOV8.

### Example 7

Experiments were conducted to examine pOV8 epitope expression over time.

[7-1]

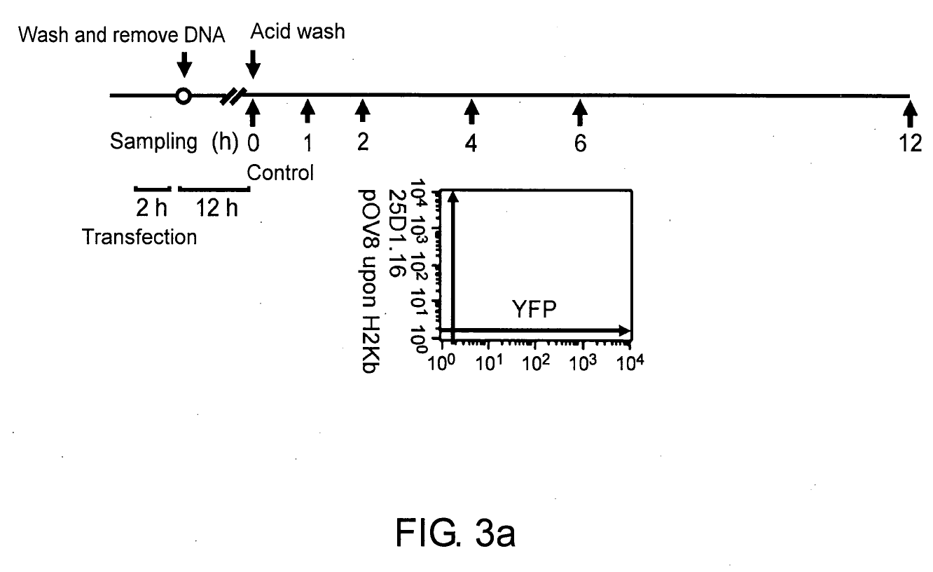

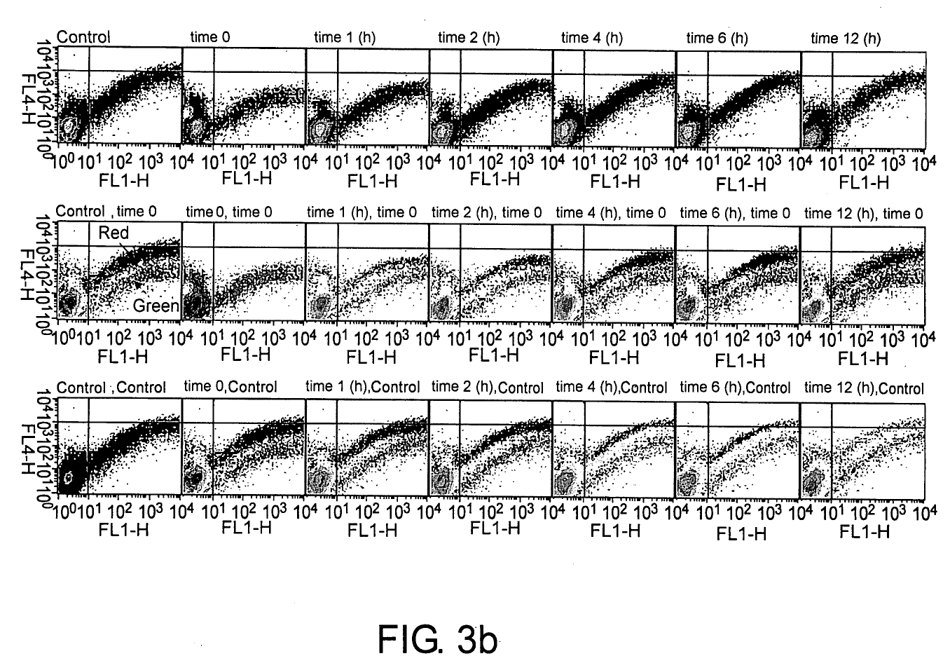

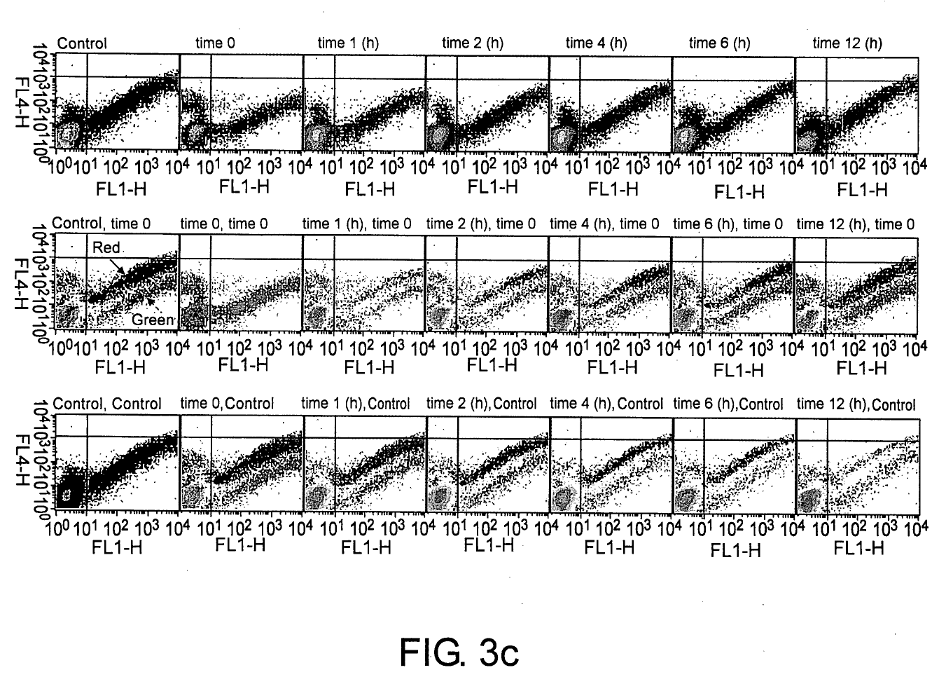

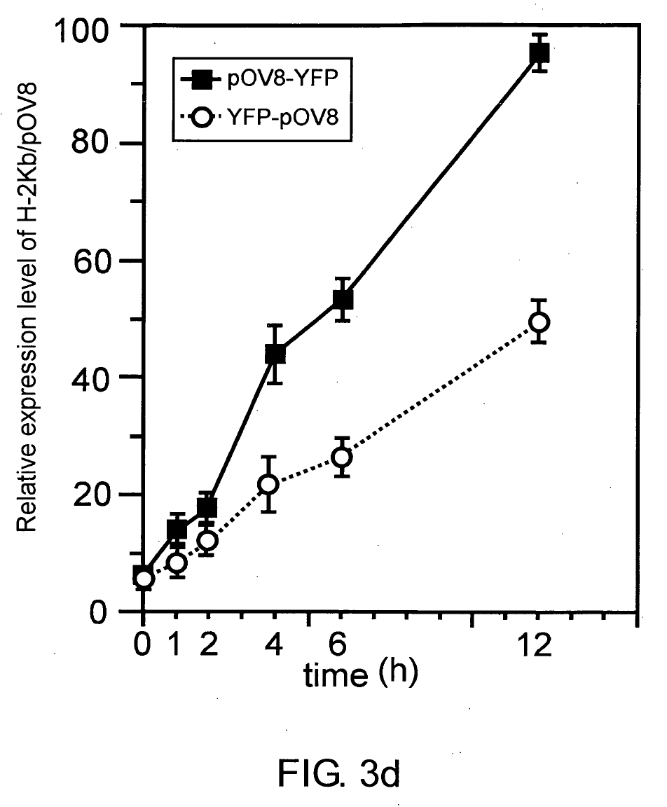

Time course is shown in FIG. 3a. DC2.4 cells were transfected with pOV8-YFP and washed after two hours to remove excess DNA. After incubating for 12 hours, the cells were washed with acid to disengage the majority of peptides binding to the H-2Kb on the cell surface (time 0). The cells were cultured under normal conditions and H-2Kb/pOV8 expression on the cellular surface was measured by the 25D1.16 antibody. Culturing was performed up to 12 hours.

The upper row in FIG. 3b shows H-2Kb/pOV8 expression. The control shows H-2Kb/pOV8 expression prior to the acid wash. The results showed that the expression level of H-2Kb/pOV8 reduced as a result of the acid wash and subsequently recovered over time.

The middle row in FIG. 3b superimposes the plot (green) at time 0 with the plot of H-2Kb/pOV8 increase over time. H-2Kb/pOV8 expression returned as the cells were cultured. The lower row in FIG. 3b shows the control (green) prior to the acid wash superimposed with the H-2Kb/pOV8 increase over time. After 12 hours of culturing, expression returned to the level prior to the acid wash.

[7-2]

The same experiment as in [7-1] above was performed using YFP-pOV8.

The time required for expression recovery in DC2.4 cells after an acid wash was shown to be basically the same as when using pOV8-YFP (FIG. 3c).

[7-3]

The recovery in H-2Kb/pOV8 expression was quantified using the results in FIG. 3b and FIG. 3c. FIG. 3d expresses relative values taking the amount of H-2Kb/pOV8 in DC2.4 cells expressing pOV8-YFP as 100%. Four hours after the acid wash, approximately 50% of H-2Kb/pOV8 expression had recovered. At 12 hours, expression had recovered to approximately the same level as the control cells. The same results were obtained for both pOV8-YFP and YFP-pOV8. These results demonstrate that pOV8 presentation to H-2Kb occurs relatively rapidly.

H-2Kb/pOV8 expression was time-dependent and, after 12 hours, was observed to recover to approximately the level prior to acid treatment. However, since the vertical axis in FIG. 3b is in logarithmic units, the expression has not in fact completely recovered (this is why the recovery curve in FIG. 3b does not show saturation).

### Example 8

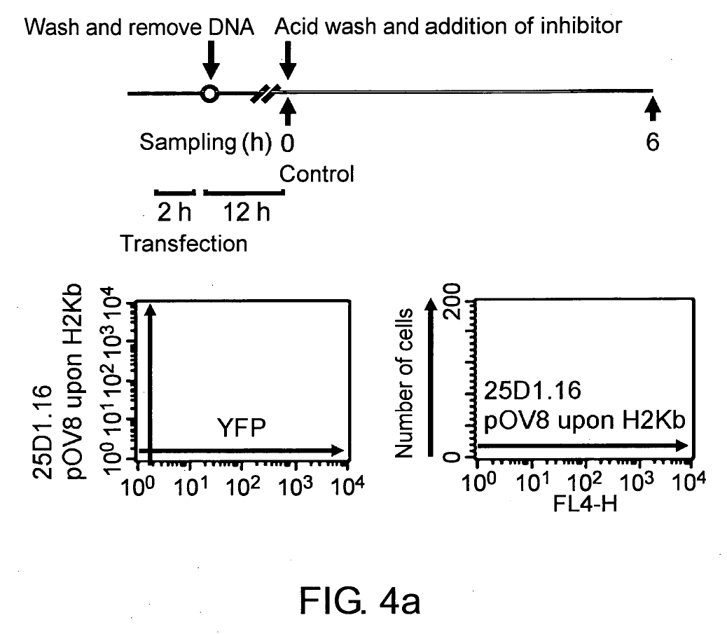

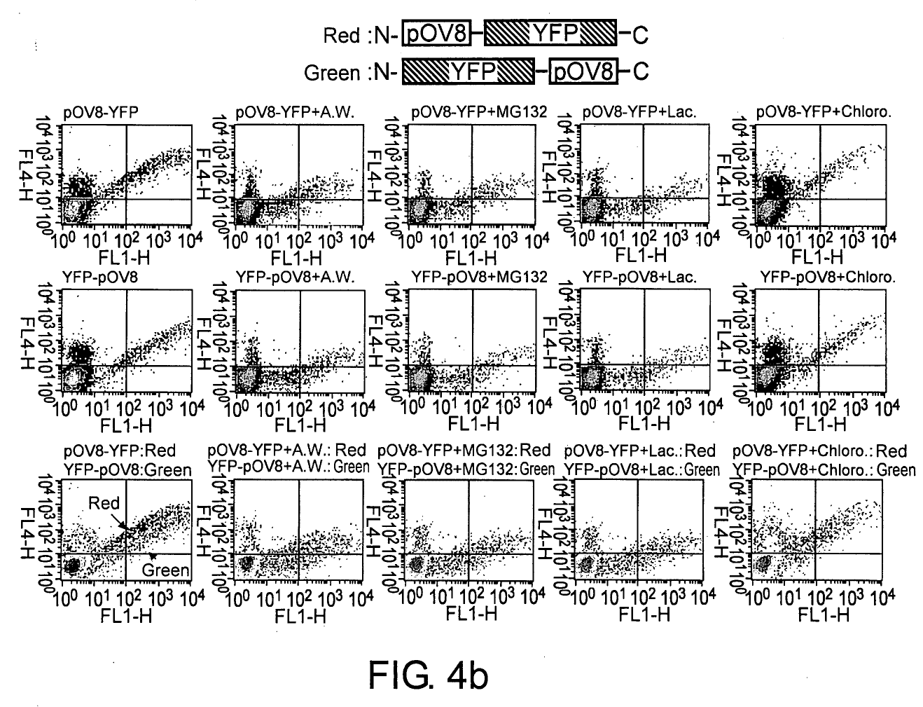

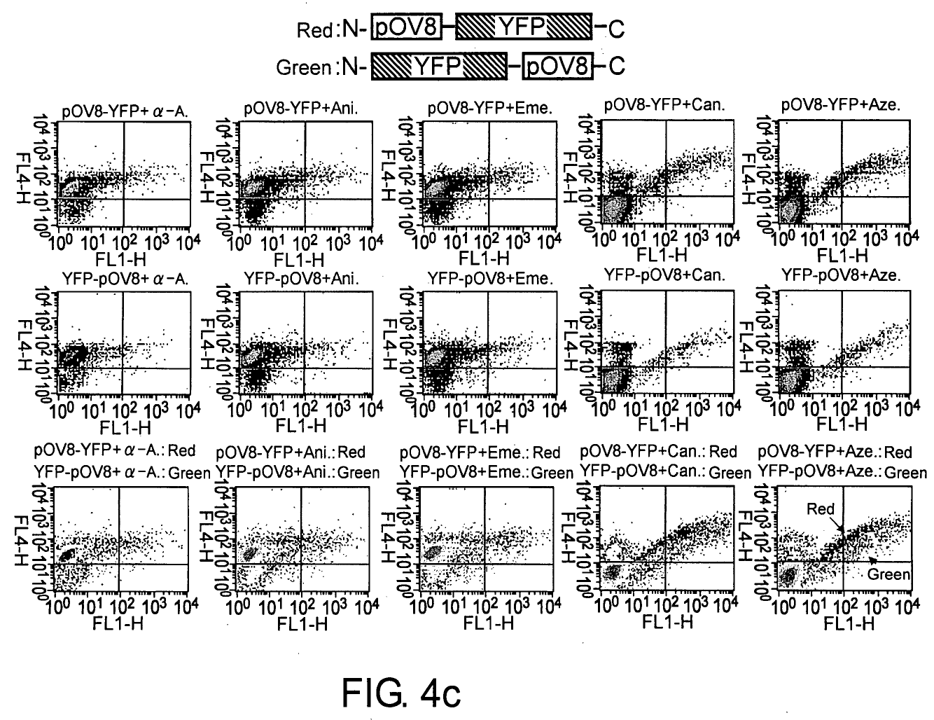

The effect of various inhibitors on H-2Kb/pOV8 expression was examined. FIG. 4a shows the time course of the experiment. DC2.4 cells were transfected with DNA and after two hours, excess DNA was removed and the cells were further incubated for 12 hours. The cells were washed with acid and the indicated inhibitor was added at the same time. After six hours, H-2Kb/pOV8 expression was measured using 25D1.16 antibody.

The upper row of FIG. 4b shows cells expressing pOV8-YFP. The results clearly indicated that H-2Kb/pOV8 presentation was inhibited by proteasome inhibitors but was not inhibited by lysosomal enzyme inhibitors (chloroquine). The middle row in FIG. 4b shows cells expressing YFP-pOV8. The lower row in FIG. 4b shows superimposition of the data from the upper and middle rows. The results showed that antigen presentation was higher with pOV8-YFP than with YFP-pOV8 even in the presence of chloroquine as in the control results, demonstrating that the N-terminal epitope dominance was maintained. These results accord with previous results showing that the cell surface expression of H-2Kb/pOV8 requires degradation of pOV8-YFP or YFP-pOV8 by proteasomes but does not require degradation by lysosomal enzymes.

### Example 9

Other inhibitors were examined as in the experiment described in [8] above.

Recovery in H-2Kb/pOV8 expression was not observed when RNA synthesis was inhibited by a-amanitin (first column from the left in FIG. 4c). When protein synthesis was inhibited using anisomycin or emetine, H-2Kb/pOV8 presentation was inhibited, although a potential antigen protein was present in the cells (second and third columns in FIG. 4c). Thus, H-2Kb/pOV8 was dependent on new RNA synthesis and protein synthesis. Specifically, it was shown that pOV8 peptides were not supplied by degradation of pOV8-YFP or YFP-pOV8 already existing in the cytosol, but rather from newly synthesized mRNA and protein (pOV8-YFP or YFP-pOV8) synthesized using that mRNA as a template.

### Example 10

The same system in [8] above was used to examine the effect of non-natural amino acids such as canavanine or azetidine. It is known that proteins incorporating non-natural amino acids are easily degraded since they fail to fold properly.

[10-1]

The effect of an amino acid analog canavanine was examined. The expression level of H-2Kb/pOV8 displayed no variation in the presence of canavanine (second column from the right in FIG. 4c) or in its absence (left column in FIG. 4c). However, even in the presence of canavanine, the level of H-2Kb/pOV8 expression on the cell surface was higher when pOV8-YFP was expressed than when YFP-pOV8 was expressed (fourth column in FIG. 4c). A full-length protein containing a non-natural amino acid is an incomplete protein in a broad sense, and is therefore thought to be easily degraded. These results showed that even an increase in the amount of incomplete protein resulting from the presence of canavanine had no effect on the principle of N-terminal epitope dominance.

[10-2]

The same experiment as in [10-1] above was conducted using azetidine instead of canavanine (right column of FIG. 4c). The presence of azetidine showed the same result that cellular surface H-2Kb/pOV8 expression was higher in cells expressing pOV8-YFP than YFP-pOV8. Furthermore, the presence or absence of azetidine had no effect on antigen presentation (data omitted).

### Example 11

The presentation of H-2Kb/pOV8 was examined when pOV8-YFP and YFP-pOV8 were introduced directly from the outside into the cytosol of DC2.4 cells.

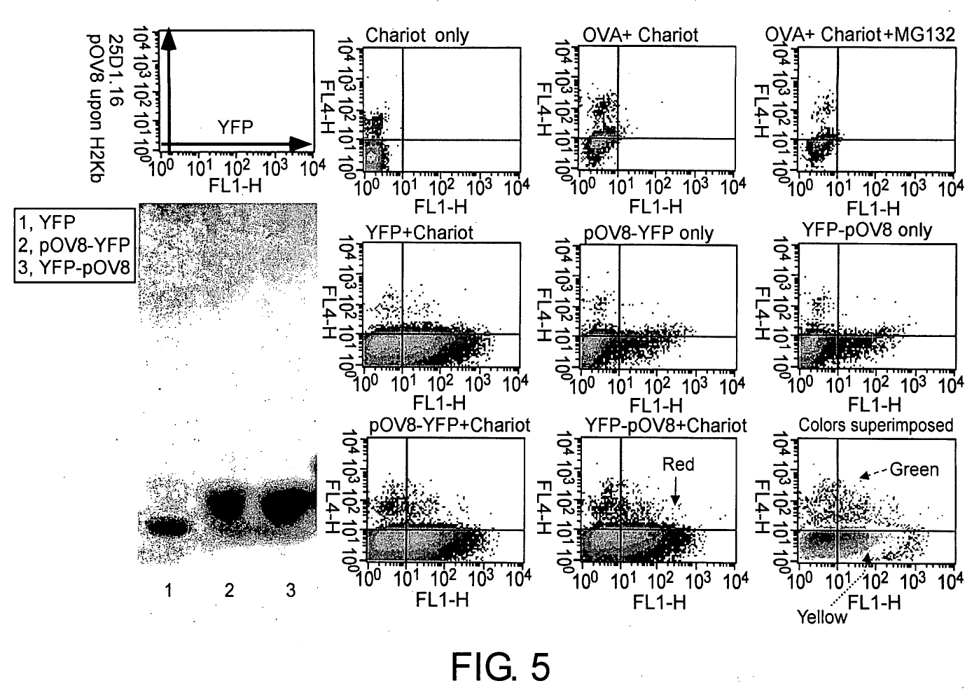

Chariot was used as a reagent for forcibly making the cell membrane permeable to proteins in solution. It was again examined whether the presentation of pOV8 to H-2Kb was improved by fusion of pOV8 to the N-terminus of YFP as compared to fusion to the C-terminus. The cells were heated in the presence of any of YFP, pOV8-YFP, and YFP-pOV8, and the cell membrane permeabilizing agent Chariot. After washing the cells, a whole cell extract was prepared. After SDS-PAGE, Western blotting was performed using anti-YFP antibody (inset of FIG. 5). In addition, the expression of H-2Kb/pOV8 in these cells was measured using the 25D1.16 antibody.

The results are shown in the inset in FIG. 5. No considerable difference was observed between the introduction of pOV8-YFP and YFP-pOV8 into the cells (compare lanes 2 and 3).

Thus, the following results were obtained.

(1) When OVA was administered to cells in the presence of Chariot, a slight presentation of H-2Kb/pOV8 was observed (FIG. 5: upper row center: a small number of plotted cells shifted upward). Administration of MG132 reduced antigen presentation (FIG. 5: upper row right: the number of cells shifting upward was decreased).
(2) When YFP and Chariot were administered, introduction of YFP into the cells was confirmed (FIG. 5: middle row left: a considerable number of cells shifted to the right).
(3) When only pOV8-YFP or YFP-pOV8 was administered externally to the cells, only a small amount of protein was introduced into the cells (FIG. 5: middle row center and right). The slight YFP fluorescence detected in the cells was thought to result from pOV8-YFP and such that underwent macropinocytosis. Furthermore, the slight level of antigen presentation occurring in the absence of the cell membrane permeabilizing agent Chariot was thought to result from the cross-presentation capability of the dendritic cells.
(4) When pOV8-YFP was administered to cells in the presence of Chariot, presentation of H-2Kb/pOV8 was observed (FIG. 5: lower row left). The same results were obtained when YFP-pOV8 was administered to cells in the presence of Chariot (FIG. 5: lower row center). There was almost no difference between the results for pOV8-YFP and YFP-pOV8 (FIG. 5: lower row right). However, higher levels of pOV8-YFP (YFP-pOV8) in cells did not result in stronger expression of H-2Kb/pOV8 (the plot does not slope upward to the right).
(5) When pOV8-YFP and YFP-pOV8 were forcibly introduced from the outside into cells, N-terminal epitope dominance was not observed.

### Example 12

The origin of N-terminal epitope dominance was examined.

Both pOV8-YFP and YFP-pOV8 are translated in the ribosomes of DC2.4 cells and expressed in the cytosol. The reason why pOV8 more easily forms complexes with H-2 kb when it is located at the N-terminus of YFP than at the C-terminus of YFP was examined.

Model 1: Since proteasomal degradation in the cytosol proceeds from the N-terminus, pOV8 release is easier when positioned close to the N-terminus rather than to the C-terminus. However, there are no experimental results supporting the model that proteasomal degradation progresses from the N-terminus to the C-terminus (on the contrary, it has been suggested that proteasomes use a sequence-specific endopeptidase action to cleave substrate proteins). According to the present study, the difference in the presentation efficiency between the N-terminus and the C-terminus depends on the protein translation process, and no differences were observed even when introducing a completely translated protein into DC2.4 cells. Accordingly, Model 1 cannot actually exist.

Model 2: Protein translation progresses from the N-terminus to the C-terminus. However, the translation of approximately 30% of the proteins translated within cells is terminated before reaching a stop codon, resulting in the release of incompletely elongated polypeptide chains from ribosomes. This is supported by several data. Since such incompletely elongated polypeptide chains lack proper tertiary structure, they are rapidly degraded by the cellular protein quality control mechanism. Such incomplete polypeptides, and full-length polypeptides that have been completely translated but have failed to form proper tertiary structure, are collectively termed DRiPs (defective ribosomal products).

DRiPs are much more rapidly degraded by proteasomes compared to complete-length proteins with normal tertiary structure (the protein quality control mechanism in the cytosol immediately degrades proteins with an abnormal structure). Furthermore, there is data suggesting that DRiPs are an extremely good source of epitopes binding to class I molecules.

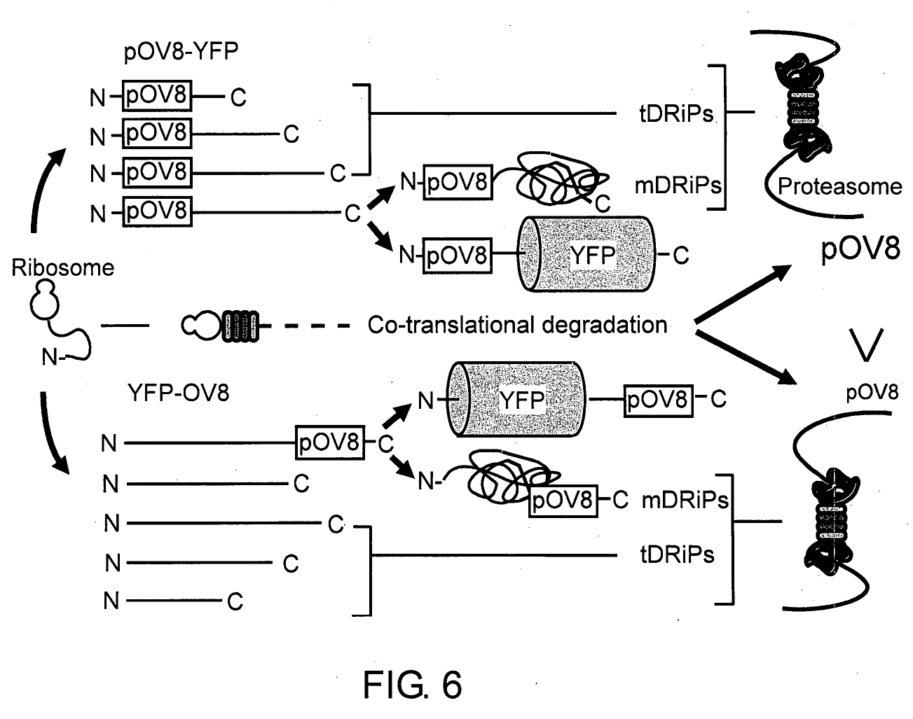

The above results suggest that the proportion of pOV8 contained in DRiPs produced by the pOV8-YFP translation process is considerably greater than the proportion of pOV8 contained in DRiPs produced by the YFP-pOV8 translation process (FIG. 6). Thus, N-terminal epitopes are contained in DRiPs in a higher proportion than C-terminal epitopes, and therefore efficiently presented by class I molecules. Therefore, Model 2 appears to provide a convincing explanation for N-terminal epitope dominance.

Model 3: There is data suggesting that substrate proteins are degraded cotranslationally (cotranslational degradation model). This model assumes that many peptides presented to MHC class I result from cotranslational degradation. According to this model, since protein synthesis is initiated from the N-terminus, it is inferred that the N-terminal portion is degraded more commonly than C-terminal portion. Therefore, Model 3 also explains N-terminal epitope dominance.

The notion that epitopes presented to class I molecules originate in partially synthesized or newly-synthesized incomplete polypeptides rather than complete proteins (having a complete tertiary structure) existing in the cells, accords well with the results of the present invention (refer to Example 9 above).

### Example 14

Experiments were conducted to check whether N-terminal dominance would be observed in reported T cell antigen epitopes actually presented on class I molecules.

First the site of T cell antigen epitopes in actual antigenic proteins was investigated. The presence of CTL or CD8⁺ cells recognizing the T cell antigen epitope-MHC 1 molecule complex has been experimentally confirmed.

[14-1]

232 T cell antigen epitopes presented on MHC class I molecules were selected from the Immuno Epitope Database. The Immuno Epitope Database stores data for partial peptides of cancer antigenic proteins. These peptides bind to MHC class I molecules and are then expressed on the surface of cancer cells. There are also CTL and CD8⁺ cells recognizing such complexes. The amino acid number from the N-terminus to each peptide (T cell antigen epitope) (vertical axis) was plotted against each peptide's affinity to the class I molecule (how easily the peptide fits into the pocket; shown by the horizontal axis). In view of the principal objective of the present Example, the horizontal axis should show the amount of antigen presentation; however, since there is no reference for standardizing different antigen epitopes, affinity to class I molecules was used).

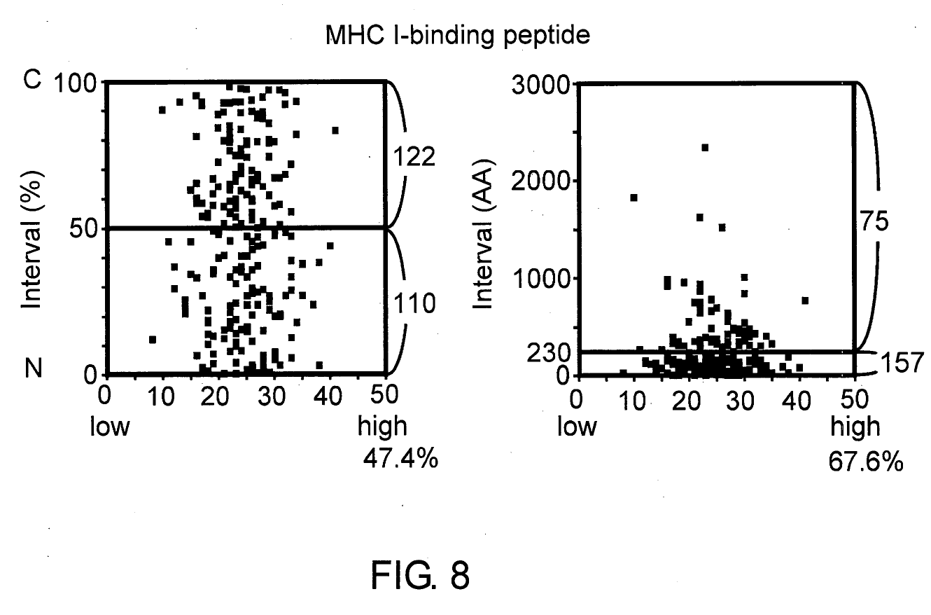

As shown on the right of FIG. 8, epitope affinity based on the database SYFPEITHI was distributed between a minimum value of 10 and a maximum of 40, with a mean of 20-30. In terms of the amino acid number from the N-terminus to the epitope, more than ⅔rd of the epitopes were concentrated within 230 residues from the N-terminus. The plot on the left in FIG. 8 shows the epitope position expressed as a relative value, which is plotted on the vertical axis where the N-terminus and the C-terminus are taken as 0% and 100%, respectively. This plot indicates that the relative position of the antigen epitope from the N-terminus in antigenic protein is completely random.

[14-2]

In addition, data on cancer immunity from the Cancer Immunity database was used. The Cancer Immunity database stores protein data and partial peptide data. The latter includes data on peptides that bind to MHC class I molecules and are expressed on various surfaces including cancer cells, and peptides that bind to MHC class I molecules (which have not confirmed to be expressed on the cell surface). Specifically, the data include T cell antigenic peptides and proteins serving as sources of T cell antigenic peptides. These T cell antigenic peptides actually form a complex with MHC class I molecules and are expressed on the cell surface. Furthermore, in many cases, the existence of CTLs (CD8⁺ cells) recognizing such complexes has also been confirmed.

T cell antigenic peptides were categorized into four types (Mutations, Shared Tumor-specific Antigens, Differentiation Antigens and Over-expressed Antigens) according to the database. In the same manner as above, the number of amino acid residues from the N-terminus of the antigenic protein to the epitope, and the affinity for class I molecules, were plotted on the vertical and horizontal axes, respectively.

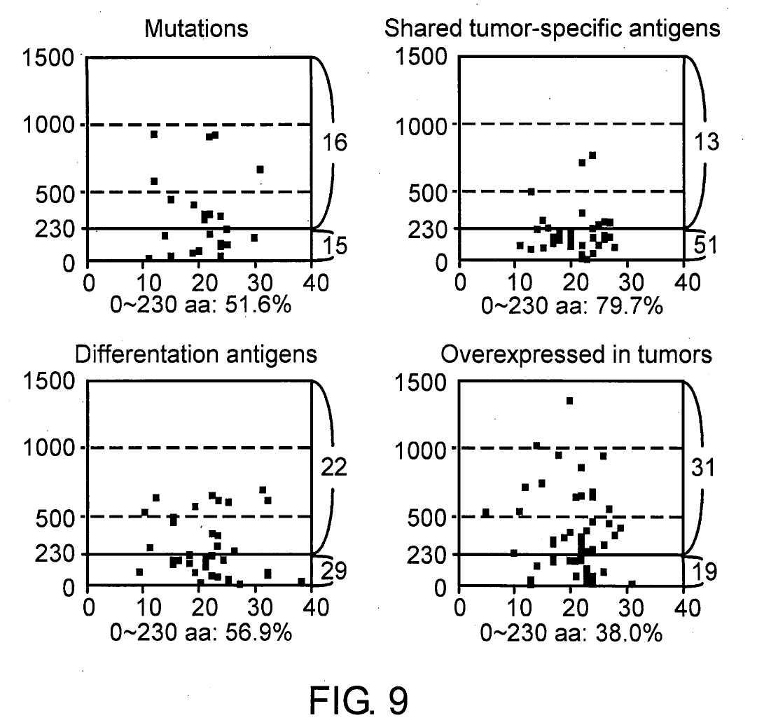

As a result, interestingly, approximately ⅔ of “Shared Tumor-specific Antigens” and “Differentiation Antigens” had epitopes located within 230 amino acid residues of the N-terminus as in the result of [14-1], while the epitopes of “Mutations” and “Over-expressed tumor antigens” were not necessarily positioned in proximity to the N-terminus (FIG. 9).

[14-3]

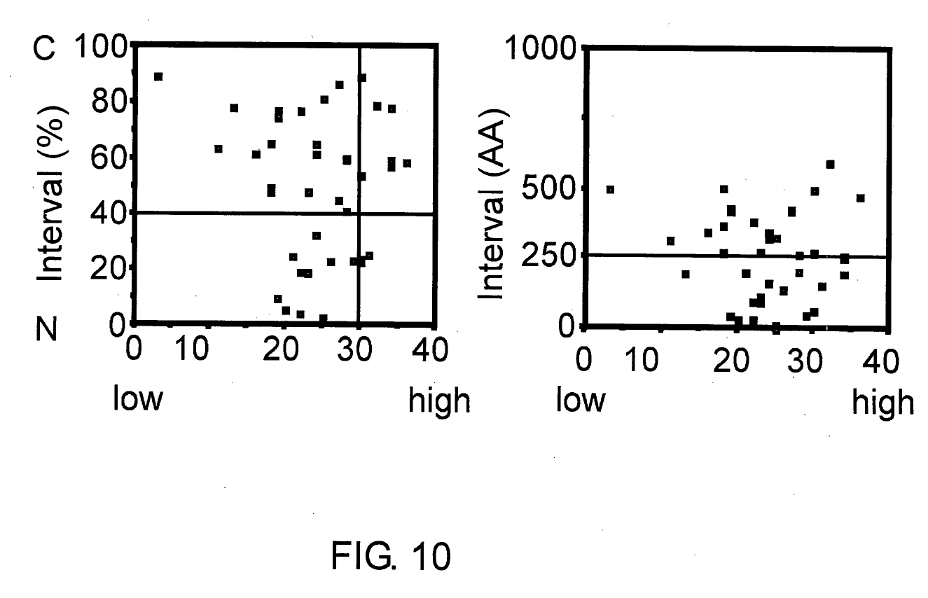

Experiments were further conducted using viral antigens. 50 viral antigen epitopes were randomly selected from the database and plotted in the same manner as above. As shown in FIG. 10, the relative position of the epitope from the N-terminus was completely random, and the number of amino acid residues was not within 230 residues of the N-terminus as seen in [14-1] above.

[14-4]

The relationship between the above results and “N-terminal epitope dominance” suggested by the experimental results of the present inventors is discussed hereafter. “Over-expressed antigens in tumors” in [14-2] and proteins providing viral antigen epitopes in [14-3] are overexpressed in cells. These overexpressed proteins are likely to include, in addition to DRiPs, many polypeptides that have been completely translated, but which are liable to be degraded as a result of incomplete folding or assembly. If this is the case, N-terminal epitope dominance cannot be applied to overexpressed proteins. On the other hand, N-terminal epitope dominance is expected to be applicable to presentation of epitopes from moderately expressed cancer antigenic proteins.

### [Discussion]

The molecular biological mechanism producing N-terminal epitope dominance was reviewed. For almost all proteins, translation is initiated from methionine in the N-terminus (recently initiation from Leu was discovered), and terminated by a stop codon in the C-terminus. In this translation process, up to approximately 30% of proteins does not complete the translation and is released from ribosomes in an incomplete form because of various reasons, such as translation errors, erroneous insertion of amino acids, translation termination prior to the stop codon, or depletion of amino acid reserves. These polypeptides are collectively referred to as DRiPs, and such polypeptides are rapidly degraded by proteasomes after release from ribosomes or while still attached to ribosomes. DRiPs may be broadly divided based on their production process into tDRiPs whose translation was prematurely terminated and mDRiPs which have completed translation, but are misfolded (FIG. 7).

Although many tDRiPs have completed N-terminal peptide synthesis, they are thought not to have completed C-terminal peptide synthesis. In contrast, mDRiPs have completed C-terminal peptide synthesis. tDRiPs, mDRiPs and mature proteins are thought to constitute the source of antigenic peptides in all cells including DCs and various types of cancer cells.

N-terminal dominance is thought to originate in tDRiPs since, in particular, only tDRiPs of the above three classes are thought to contain almost no C-terminal antigenic peptides (FIG. 7). On the other hand, it may be possible to explain that “N-terminal dominance” is merely appearance-only, based on the assumption that the fluorescence per molecule of the fluorescent protein fused with an antigen peptide at the N-terminus is weaker than the fluorescence per molecule of the fluorescent protein fused with an antigen peptide at the C-terminus. However, if this assumption is correct, when the scatter plot of the cells is developed only using the fluorescence intensity of the fluorescent protein, there should be a large difference between the fusion fluorescent protein at the N-terminus and the fusion fluorescent protein at the C-terminus. In fact, however, the expected difference was not observed. In addition to the fact that almost no difference was evident when GST-YFP and YFP-GST were compared, N-terminal dominance was also confirmed when using three types of fluorescent proteins, Kaede, Kikume Green-Red, Kusabira Orange in addition to YFP and Azamigreen shown herein. Since the tertiary structure of these five fluorescent proteins is different, it is difficult to think that N-terminal fusion would cause more loss of fluorescent intensity than C-terminal fusion across all five fluorescent proteins. This conclusion rejects the assumption that the fluorescence per molecule of the fluorescent protein fused with an antigenic peptide at the N-terminus is weaker than the fluorescence per molecule of the fluorescent protein fused with an antigenic peptide at the C-terminus.

The most distinguishing feature of the model proposed by the present inventors is that tDRiPs and mDRiPs are both byproducts of protein synthesis and have no relationship to the total amount of completely synthesized proteins or to protein stability. Consequently, it is suggested that N-terminal dominance entirely depends on the translation process and therefore may be a general rule.

In addition, the inventors employed YFP and fusion proteins thereof as model proteins in a major part of the present invention. YFP and the YFP fusion proteins used in the present invention are both extremely stable proteins (FIG. 7). Consequently this suggests that throughout the course of the experiments conducted by the present inventors, there were almost no antigenic peptides originating from the mature protein. Furthermore, the experiment results for inhibition of translation (FIG. 4) and introduction of mature proteins into the cytoplasm (FIG. 5) suggest that the amount of antigenic peptides originating from mature proteins is extremely small and that no difference in antigen presentation is caused between mature fusion proteins having an antigenic peptide at the N-terminus and mature fusion proteins having an antigenic peptide at the C-terminus. While the inhibition of translation (FIG. 4) and introduction of mature proteins into the cytoplasm (FIG. 5) completely eliminate the production of DRiPs, they do not affect the degradation of mature proteins at all. Therefore, these results strongly support the hypothesis of the present inventors that N-terminal dominance originates in degradation of DRiPs.

Another characteristic of DRiPs is their rapid proteasomal degradation. Thus, inhibition of proteasomes completely inhibits antigen presentation (FIG. 4). In contrast, the inhibition of proteases in endosomes/lysosomes promoted antigen presentation. This suggests that although the majority of DRiPs are degraded by proteasomes, a part thereof may be degraded by proteases in endosomes/lysosomes.

Searches of databases for already existing MHC I antigen epitopes obtained results supporting N-terminal dominance. Cancer-derived MHC I epitopes with confirmed immunological effects were selected from databases, and the number of amino acids from the first methionine of the protein to the N-terminus of the epitope was plotted on the Y axis and the affinity of MHC I epitope on the X axis (FIG. 8). The results showed that a large cluster of cancer-derived MHC I epitopes was located in a region (horizontal axis score of 10-30) of relatively strong affinity to MHC I. Assuming that the average amino acid length of all proteins is 460 amino acids, 68% of MHC I epitopes are in the N-terminal side (from the 230th amino acid to the N-terminus) of the protein. Meanwhile, signal peptides are known to be on the N-terminal side of the protein and easily transformed into MHC I epitopes. These results support N-terminal dominance for T cell antigen epitopes.

T cell antigen epitopes of viral origin were then plotted in the same manner for contrast purposes (FIG. 10). Although virus-derived T cell antigen epitopes were concentrated in regions (horizontal axis score of 20-40) with even higher affinity for MHC I, a concentration to the N-terminal side was not observed at all. Thus, N-terminal dominance is not present in virus-derived T cell antigen epitopes. The difference between cancer-derived T cell antigen epitopes and virus-derived T cell antigen epitopes may be explained as follows. The CD8⁺ cell selection mechanism in the thymus gland can explain that viral T cell antigens show a particularly strong affinity for MHC I while cancer T cell antigens do not. More specifically, cancer-specific proteins are intrinsically expressed in small amounts in healthy individuals and should have some type of function. Therefore, if there is a cancer-derived T cell antigenic epitope having an extremely strong affinity to MHC I and CD8⁺ cells recognizing the epitope, the cells will be removed as clones reacting against self (or will be led to an anergic state). If they are not removed, an autoimmune disease will result. However, at present there is no clear explanation for why N-terminal dominance is not seen in the production of viral epitopes having extremely strong affinity to MHC I. The above results demonstrate that the selectivity of peptides presented on MHC I clearly depends to a large extent on the position of the peptides on the protein in addition to the classical principles such as the affinity of peptides to MHC I, expression level and protein stability.

1. A method for determining whether a test peptide is a candidate T cell antigen peptide presented on the surface of a cancer cell following binding to an MHC class I molecule, which comprises the steps of:
(a) providing a test T cell antigen peptide that binds to an MHC class I molecule; and
(b) determining the position of amino acid sequence of said T cell antigen peptide provided in step (a) in the amino acid sequence of a protein comprising the peptide;
wherein, when the position of the amino acid sequence determined in step (b) is within 230 amino acids from the N terminus of the amino acid sequence of the protein, the test peptide is determined to be a candidate T cell antigen peptide presented on the surface of a cancer cell following binding to the MHC class I molecule.

2. A method for selecting a candidate T cell antigen peptide presented on the surface of a cancer cell following binding to an MHC class I molecule, which comprises the steps of:
(c) determining by the method of claim 1 whether each of several test peptides is a candidate T cell antigen peptide presented on the surface of a cancer cell following binding to an MHC class I molecule; and
(d) selecting a test peptide that has been determined in step (c) to be a candidate T cell antigen peptide to be presented on the surface of a cancer cell through binding to an MHC class I molecule.

3. The method of claim 1 or 2, wherein the test peptide is a partial peptide of a cancer-specific protein.

4. A method for selecting a candidate T cell antigen peptide presented on the surface of a cancer cell following binding to an MHC class I molecule, which comprises the steps of:
(e) performing an algorithm for predicting MHC class I molecule-binding peptides on the amino acid sequence of a desired protein to identify the amino acid sequence of a peptide that is predicted to bind to an MHC class I molecule;
(f) determining the position of the amino acid sequence identified in step (e) in the amino acid sequence of the protein; and
(g) selecting a peptide comprising the amino acid sequence whose position determined in step (f) is within 230 amino acids from the N terminus of the amino acid sequence of the protein.

5. A method for identifying a candidate T cell antigen peptide presented on the surface of a cancer cell following binding to an MHC class I molecule, which comprises the step of performing an algorithm for predicting MHC class I molecule-binding peptides on an amino acid sequence positioned within 230 amino acids from the N terminus of a desired protein to identify the amino acid sequence of a peptide that is predicted to bind to an MHC class I molecule, wherein a peptide comprising the amino acid sequence identified in said step is identified as a candidate T cell antigen peptide presented on the surface of a cancer cell following binding to an MHC class I molecule.

6. The method of claim 4 or 5, wherein the desired protein is a cancer-specific protein.

7. A method for identifying a candidate T cell antigen peptide that can be used as a cancer vaccine, which comprises the steps of:
(h) identifying a cancer-specific protein;
(i) identifying the amino acid sequence of a peptide that is predicted to be presented on the surface of a cancer cell through binding to an MHC class I molecule of the same haplotype as that of a patient subjected to cancer treatment or prevention, from the amino acid sequence of the protein identified in step (h);
(j) contacting a peptide comprising the amino acid sequence identified in step (i) with an MHC class I molecule of the same haplotype as that of said subject to form a complex;
(k) contacting the complex formed in step (j) with a CD8-positive T cell isolated from said patient; and
(l) detecting the binding of the complex formed in step (j) to the CD8-positive T cell isolated from the patient;
wherein, when the binding is detected in step (l), a peptide comprising the amino acid sequence identified in step (i) is identified as a candidate T cell antigen peptide that can be used as a cancer vaccine.

8. A method for identifying a candidate T cell antigen peptide that can be used as a cancer vaccine, which comprises the steps of:
(m) identifying a cancer-specific protein;
(n) performing an algorithm for predicting MHC class I molecule-binding peptides on an amino acid sequence positioned within 230 amino acids from N terminus of the protein identified in step (m) to identify the amino acid sequence of a peptide that is predicted to bind to an MHC class I molecule of the same haplotype as that of a patient subjected to cancer treatment or prevention;
(o) contacting a peptide comprising the amino acid sequence identified in step (n) with an MHC class I molecule of the same haplotype as that of said patient to form a complex;
(p) contacting the complex formed in step (o) with a CD8-positive T cell isolated from the patient; and
(q) measuring the binding of the complex formed in step (o) to the CD8-positive T cell isolated from the patient;
wherein, when the binding is detected in step (q), a peptide comprising the amino acid sequence identified in step (n) is identified as a candidate T cell antigen peptide that can be used as a cancer vaccine.

9. A method for identifying a candidate T cell antigen peptide that can be used as a cancer vaccine, which comprises the steps of:
(r) identifying a cancer-specific protein;
(s) performing an algorithm for predicting MHC class I molecule-binding peptides on the amino acid sequence of the protein identified in step (r) to identify the amino acid sequence of a peptide that is predicted to bind to an MHC class I molecule of the same haplotype as that of a patient subjected to cancer treatment or prevention;
(t) determining the position of the amino acid sequence identified in step (s) in the amino acid sequence of the protein identified in step (r);
(u) selecting an amino acid sequence whose position determined in step (t) is within 230 amino acids from the N terminus of the amino acid sequence of the protein identified in step (r), as the amino acid sequence of a peptide to be presented on the surface of a cancer cell through binding to an MHC class I molecule of the same haplotype as that of the patient;
(v) contacting a peptide comprising the amino acid sequence selected in step (u) with an MHC class I molecule of the same haplotype as that of the patient to form a complex;
(w) contacting the complex formed in step (v) with a CD8-positive T cell isolated from the patient; and
(x) detecting the binding of the complex formed in step (v) to the CD8-positive T cell isolated from the patient;
wherein, when the binding is detected in step (x), a peptide comprising the amino acid sequence selected in step (u) is identified as a candidate T cell antigen peptide that can be used as a cancer vaccine.
